(19) United States
(12) Patent Application Publication
(10) Pub. No.: US 2011/0229517 A1
(43) Pub. Date: Sep. 22, 2011

(54) SYSTEM AND PROCESS FOR PRODUCING MULTI-COMPONENT BIOPHARMACEUTICALS

(75) Inventors: Kirsten Strahlendorf, Toronto (CA); Kevin Harper, Cheltenham (CA)

(73) Assignee: SANOFI PASTEUR, LTD., Toronto (CA)

(21) Appl. No.: 13/050,736

(22) Filed: Mar. 17, 2011

Related U.S. Application Data
(60) Provisional application No. 61/314,864, filed on Mar. 17, 2010.

(51) Int. Cl. A61K 39/00; A61K 39/12; A61K 39/02; A61P 37/04; A61P 31/04; A61P 31/10; A61P 31/12; A61P 33/00; A61P 35/00; B01J 19/00

(52) U.S. Cl. 424/204.1; 424/184.1; 424/234.1; 424/274.1; 424/265.1; 424/277.1; 422/291

(57) ABSTRACT

A sterile, closed, disposable system for formulating biopharmaceutical compositions containing multiple active agents is described herein.

### RELATED APPLICATIONS

This application claims priority to U.S. Ser. No. 61/314,864 filed Mar. 17, 2010 and Canadian Pat. Appln. No. 2,697,804 filed Mar. 17, 2010.

### FIELD OF STUDY

This disclosure relates to devices and methods for preparing multi-component biopharmaceutical formulations within a closed manufacturing system.

### BACKGROUND

Biopharmaceutical formulations often consist of multiple active ingredients within a single composition. Vaccines are one of the most familiar product types that comprise multiple biological and non-biological components in single formulation. Those skilled in the art often encounter challenges in preparing such formulations including system clogging, inaccuracies, and low binding of active ingredients. Such problems may be overcome by using a closed, disposable system (e.g., “single use”). Thus, there is a recognized need in the art for such a system. Single-use processing has major advantages over conventional methods, such as a lower potential for contamination (e.g., particulates and bioburden), reduced capital expenditure, and elimination of in-house cleaning and sterilization steps. Exemplary systems are described below.

### SUMMARY OF THE DISCLOSURE

Described herein are sterile, closed, disposable systems for formulating a biopharmaceutical composition comprising multiple active agents. The system typically includes multiple components (or parts) linked in series, the components typically being one or more buffer reservoirs; multiple reservoirs of active agents, each reservoir containing a different active agent or combination of active agents; one or more pumps; one or more sterilizing filters; and, a station for mixing formulations comprising multiple active agents with one another. The station typically includes a final bulk formulation reservoir, optionally, at least one auxiliary reservoir containing one or more additional components and at least one pump for combining the contents of each active agent reservoir and the auxiliary reservoir in the final bulk formulation reservoir. In other embodiments, the station may include one or more of at least one intermediate formulation reservoir corresponding to each of the active agent reservoirs, optionally, at least one auxiliary reservoir containing one or more additional components, at least one pump for combining in each intermediate formulation reservoir the contents of the corresponding active agent reservoir with the contents of the auxiliary reservoir and for subsequently combining the contents of each intermediate formulation reservoir in a final formulation reservoir. In certain embodiments, one or more additional components may also be added to form a final formulation. In some embodiments, the active agent may be an antigen and/or the one or more additional components may be one or more adjuvants. Other embodiments are described in and/or may be derived from the description provided herein.

### BRIEF DESCRIPTION OF THE DRAWINGS

FIG. 1. Exemplary sterile, closed, disposable system.

FIG. 2. Exemplary system for blending intermediate formulations into a final formulated bulk bag.

FIG. 3. Exemplary sterile, closed, disposable system.

FIG. 4. Exemplary system for blending intermediate formulations into a final formulated bulk bag.

FIG. 5. The “Scenario 2” process, where proteins are filtered while being added to the formulation bag, diluted and alum-adjuvanted.

FIG. 6. “Scenario 1, Part A”, where proteins are first individually adjuvanted.

FIG. 7. “Scenario 1, Part B”, where proteins are filtered, individually adjuvanted and diluted.

FIG. 8. Exemplary filtration assembly.

FIG. 9. Study CA-08-077, Scenario 1, Part A: Formulation and Sampling Assembly.

FIG. 10. Study CA-08-077, Scenario 1, Part B: Trivalent (adj) Formulation and Sampling Assembly.

FIG. 11. Study CA-08-077-C, Scenario 2: (Unadjuvanted) Proteins added to formulation system while filtered.

FIG. 12. Multivalent Broth Formulation Assembly. The manifold on the left represents the lines from each antigen, buffer and excipient used in the process.

FIG. 13. Exemplary sterile, closed, disposable system showing case (A) holding pinch valve assemblies, the control system (B) for reading inputs and feed outputs, and the load cell control panel and display (C).

FIG. 14. Exemplary sterile, closed, disposable system with pinch valves (1A, 1B, 1C, 2A, 2B, 2C, 3A, 3B, 3C, 4A, 4B, 4C).

FIG. 15. Exemplary container.

FIG. 16. Exemplary system.

FIG. 17. Trivalent Adjuvanted formulation Process in Closed Disposable Assembly.

### DETAILED DESCRIPTION

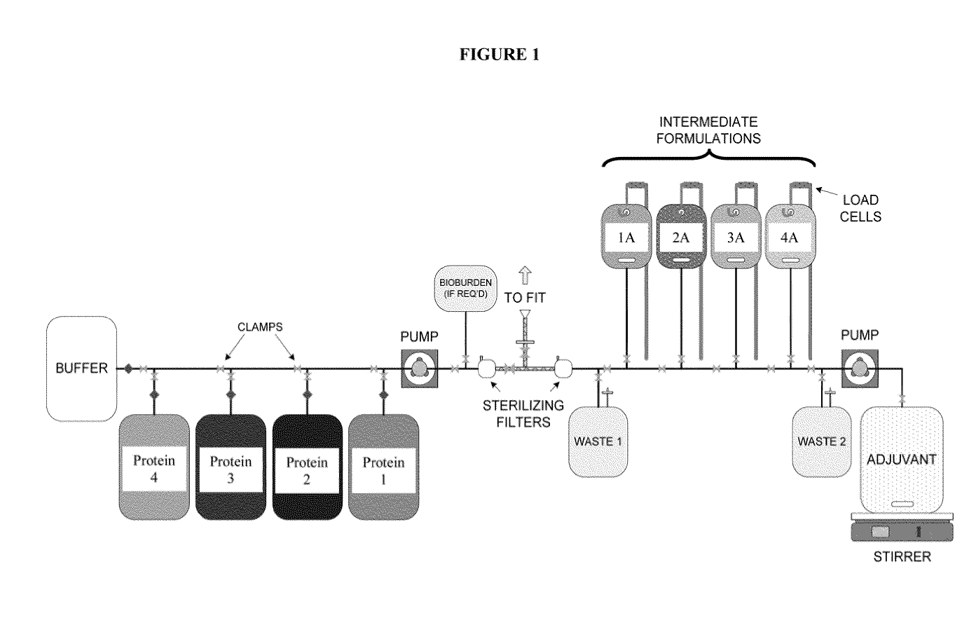

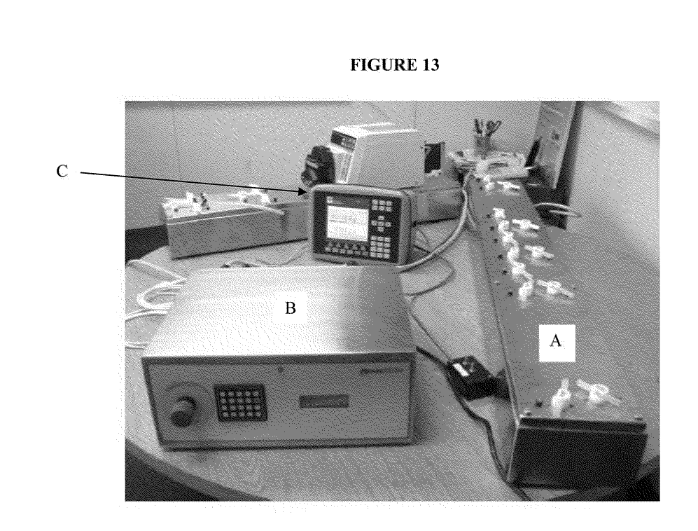

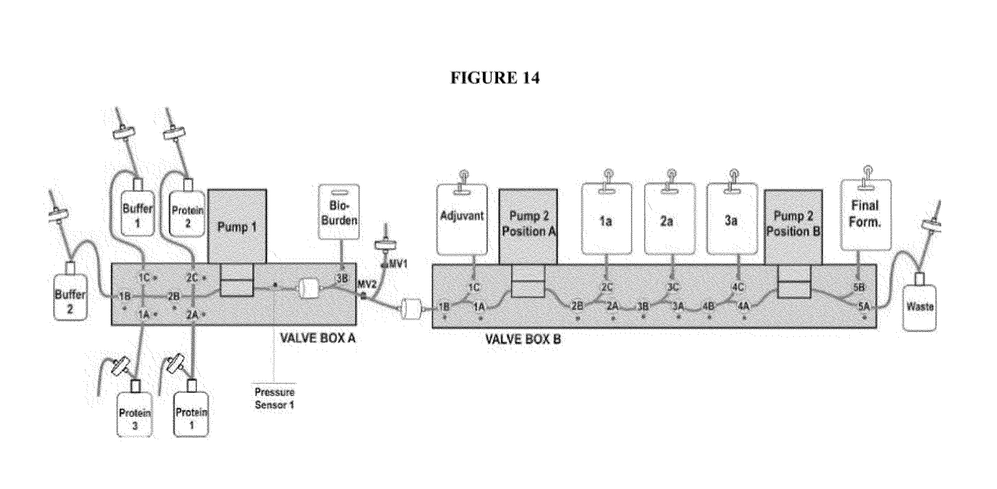

A sterile, closed, disposable system for formulating biopharmaceutical compositions containing multiple active agents is described herein. The system typically includes multiple components (or parts) linked in series, the components typically being one or more buffer reservoirs; multiple reservoirs of active agents, each reservoir containing a different active agent or combination of active agents; one or more pumps; one or more sterilizing filters; and, a station for mixing the multiple active agents with one another. The station typically includes a final bulk formulation reservoir, optionally, at least one auxiliary reservoir containing one or more additional components and at least one pump for combining the contents of each active agent reservoir and the auxiliary reservoir in the final bulk formulation reservoir. In some embodiments, the station may include one or more of at least one intermediate formulation reservoir corresponding to each of the active agent reservoirs, optionally, at least one auxiliary reservoir containing one or more additional components, at least one pump for combining in each intermediate formulation reservoir the contents of the corresponding active agent reservoir with the contents of the auxiliary reservoir and for later combining the contents of each intermediate formulation reservoir in a final formulation reservoir. In certain embodiments, the system includes a buffer reservoir; multiple reservoirs of active agents, each reservoir containing a different active agent or combination of active agents; one or more pumps; one or more sterilizing filters; multiple single-use, pre-sterilized bags, each bag containing a formulation of an active agent or combination of active agents corresponding to those in the reservoirs; and, a station for mixing the formulations contained within the bags with one another to produce a final formulation, where these parts are operably linked to one another in series. The system may also comprise one or more reservoirs for waste materials. Any or all of these parts may comprise the system described herein. In certain embodiments, one or more additional components (e.g, an adjuvant) may also be added to form a final formulation. In some embodiments, the active agent may be an antigen and/or the one or more additional components may be one or more adjuvants. Other embodiments are described in and/or may be derived from the description provided herein (e.g., including any of FIGS. 1-17).

In some embodiments, a sterile, closed, disposable system for formulating a biopharmaceutical composition comprising multiple active agents is provided, the system typically having: one or more buffer reservoirs; multiple reservoirs of active agents, each reservoir containing a different active agent or combination of active agents; one or more pumps; one or more sterilizing filters; a station for mixing the formulations with one another, the station comprising: at least one intermediate formulation reservoir corresponding to each active agent reservoir; optionally at least one auxiliary reservoir containing one or more additional components; at least one pump for combining the contents of each load cell and the auxiliary reservoir in a final bulk formulation reservoir; wherein the reservoirs are operably linked to one another in series. In certain embodiments, the reservoirs may be single-use, pre-sterilized bag(s). In some embodiments, the system may comprise one or more of: at least two sterilizing filters; a bioburden container positioned between the at least two sterilizing filters; a waste container positioned between the at least one sterilizing filter and the final bulk formulation reservoir; a waste container positioned at the end of the process line after the final station. In some embodiments, one or more of the stations is not fixably attached to a support surface. In certain embodiments, including some preferred embodiments, each active agent is an antigen and the one or more additional components is an adjuvant.

In some embodiments, a sterile, closed, disposable system for formulating a biopharmaceutical composition comprising multiple active agents, the system typically having: one or more buffer reservoirs; multiple reservoirs of active agents, each reservoir containing a different active agent or combination of active agents; one or more pumps; one or more sterilizing filters; a station for mixing the multiple active agents with one another, the station comprising: a final bulk formulation reservoir; optionally, at least one auxiliary reservoir containing one or more additional components; at least one pump for combining the contents of each active agent reservoir and the auxiliary reservoir in the final bulk formulation reservoir; wherein the parts are operably linked to one another in series. In certain embodiments, the system may also comprise a station for mixing formulations, the station comprising: at least one intermediate formulation reservoir corresponding to each of the active agent reservoirs; optionally, at least one auxiliary reservoir containing one or more additional components; and, optionally, at least one pump for combining in each intermediate formulation reservoir the contents of the corresponding active agent reservoir with the contents of the auxiliary reservoir and for later, combining the contents of each intermediate formulation reservoir in a final formulation reservoir; wherein the parts are operably linked to one another in series. In some embodiments, the systems described herein may include one or more of: a single-use, pre-sterilized bag; operable linkage between reservoirs using pre-sterilized tubing; at least two sterilizing filters; at least one bioburden container positioned between the at least two sterilizing filters; a waste container positioned between the at least one sterilizing filter and the final bulk formulation reservoir or at the end of the process line; a reservoir that is not fixably attached to a support surface; a system in which each active agent is an antigen and the one or more additional components is an adjuvant.

Also provided are sterile, closed systems comprising one or more first reservoirs containing the same or different buffers, one or more second reservoirs containing the same or different antigens, at least one third reservoir containing at least one adjuvant, the reservoirs being linked in series wherein the at least one third reservoir terminates the series and samples from each reservoir are combined to form an immunogenic composition. The reservoirs in these systems may be comprised of a single-use, disposable material.

In some embodiments, methods for preparing a multi-component biopharmaceutical composition comprising combining multiple active agents from individual active agents contained in individual reservoirs after passing the contents of each reservoir through at least one sterilizing filter, combining the components of each reservoir into an intermediate formulation within a container containing one or more additional components, and combining the contents of each container into a final formulation comprising all active agents and additional components using the systems described herein are provided. The methods may be used to produce immunogenic compositions (e.g., vaccines) using one or more antigens derived from a source selected from the group consisting of one or more viruses, bacterial species, fungal species, parasitic species, and/or tumor cell.

The process typically begins with a concentrated, purified active agent (e.g., protein) and ends with a sterile, filtered, final formulated bulk ready for sterile connection to a filling line (e.g., for a vaccine). The process may include, for example, mixing a purified protein with a buffer and/or excipient, filtering the mixture and optionally adding adjuvant to form an intermediate stock solution, optionally adding additional buffer and/or excipient as needed, and blending the intermediate stock solutions to form a final bulk formulation. Thus, in some embodiments, a pre-filter integrity test, a double (e.g., 2×) sterile filtration of concentrated purified protein(s) (e.g., active agent), and the addition of one or more buffers (and optionally one or more excipients) is performed. Each protein may be added to an intermediate bag following a thorough flushing of the lines with buffer. Proteins may be adjuvanted in these bags, with one intermediate bag dedicated to each protein, and mixed to ensure adequate binding activities. For instance, proteins may be adsorbed to an aluminum adjuvant (e.g., aluminium phosphate (AlPO₄), aluminum hydroxide (AlOOH), phosphate-treated AlOH) to nearly 100% or 100%. A post-filter integrity test may then be performed on the final filter. The next step may involve combining the individual adjuvanted proteins from the intermediate bags into a final 5 L formulation bag. In alternative embodiments, the individual proteins may be added to the final formulation reservoir and mixed together. Optionally, the components of any auxiliary reservoir (e.g., comprising an adjuvant) may be added to the formulation reservoir (e.g., before or after individual proteins have been added). The blended formulation may then be further diluted with adjuvant top-up to achieve desired concentrations within the multi-component biopharmaceutical formulation. In one embodiment, the process provides for a vaccine formulation comprising multiple antigens, at least one adjuvant, and buffers and/or excipients in the final product. The stages of the process preferably include, for example, filtration, intermediate formulation, final formulation, and blending (FIGS. 1-4). Other embodiments are also contemplated.

The parts of the system described herein are typically operably linked to one another in series to provide a closed system for producing a multi-component biopharmaceutical formulation. For example, the system may include a buffer reservoir linked to multiple reservoirs of active agents, each reservoir containing a different active agent or combination of active agents, which may be driven through a sterilizing filter using a pump, and then into one or more optional single-use, pre-sterilized bags that may optionally contain additional components (e.g., one or more adjuvants) that may each contain a formulation of an active agent or combination of active agents with or without one or more additional components, and then into a single container (which may contain one or more additional components (e.g., one or more adjuvants)) linked to a station for mixing the formulations contained within the bags with one another. In this way, multiple active agents and/or additional components may be mixed into a single formulation. The system is useful for preparing a wide variety of multi-component biopharmaceutical products (e.g., containing multiple active agents). This system may combine, for example, ingredient addition (e.g., buffers, active agents, additional components such as adjuvants), filtering, and blending into sterile, operably linked processing lines and, in particular, assemblies. A particular advantage of this system is that the process is contained to maintain sterility. The process is based on displacement of fluid in the lines, in-process measurement in the bags during addition, and filtration of multiple components through the same filtration assembly before mixing. Another advantage is that the reservoirs, bags, tubing and other materials may be disposable. The reservoirs, bags and tubing and other materials that contact stock solutions (e.g., containing active agents) are typically manufactured of a material that is not reactive with the active agent such that the active agent maintains its integrity when stored therein.

The system described herein also typically contains one or more pumps. For example, the system may include one or more peristaltic pumps. Suitable pumps include but are not limited to Masterflex or Watson-Marlow pumps or any other pump known to one skilled in the art. As for the reservoirs described above, the single-use, pre-sterilized bags for containing a formulation of an active agent or combination of active agents corresponding to those in the reservoirs are typically manufactured of a material that in not reactive with the active agent such that the active agent maintains its integrity when stored therein. Exemplary materials are readily available in the art.

The disposable items are preferably gamma sterilized and assembled using a sterile connection device such as a tube welder or Kleenpak® connector. To prevent multiple sterile filter integrity testing, taking up to 15 minutes per filter, a 2× sterile filtration assembly has been designed and may be utilized to filter all filterable components in one closed, single-use process. Using hanging load cell technology and movement of fluid in the lines by displacement, components may be dispensed accurately into the bags. The dispensing volumes by weight may be calculated using known concentration values, specific gravity, and an expected final formulation volume.

It is preferred that the system or parts of the system are maintained in a sterile, closed environment without direct contact with the formulation unless the system or part of the system is also sterile, preferably single use, and maintains the sterile liquid pathway of the closed system assembly. A single-use system offers flexibility to such changes, allows faster scale-up compared with standard technologies (stainless steel counterparts) (Cardona and Allen, 2006), and provides cost advantages. Exemplary single-use assemblies (e.g., as shown in the Examples) consist of two (2) and three (3) D bags connected to a manifold of tubing, connectors, and filters but variations are also possible. The bags used in the Examples were custom-made by the bag manufacturer, assembled, sealed into bags, and gamma-irradiated using a validated sterilization method. The films and tubings used in the systems described herein preferably exhibit inert compatibility properties, gamma-irradiation stability, quality testing, biological safety testing, and low leachables/extractables profile (Cardona and Allen, 2006). The films and tubings utilized in the system are preferably consistent for each component. The bags, tubing and filters are supported by stands and holding apparatuses assuring proper alignment and dispensing control for the connections. The system also typically includes reinforced tubing to meet pressure requirements for inline filter integrity testing of the liquid sterilizing grade filters (Cardona and Allen, 2006). In both pre- and post-integrity testing, after flushing the final filter with buffer, compressed air may be applied from the filter tester, connected in-line, through a 0.2-μm vent filter just upstream of the final filter.

For a disposables filtration and formulation design, the user will typically consider and adjust as necessary the chemical composition of the active agent or other components utilized, the concentration thereof, pH, viscosity, solubility, particle size, osmolarity, ionic strength, surfactant addition, shear sensitivity, specific gravity, product internal reactions (desired or undesired), and inter-component compatibility prior to manufacturing (Cardona and Allen, 2006; Motzkau and Okhio, 2005; Luckiewicz, 2004). Dispensing volumes by weight may be calculated using known concentration values, specific gravities, and an expected final formulation volume. Setup is typically performed with processing liquids that have fluid properties similar to water including density, viscosity, and pH (physiological). Once the proteins and other constituents are primed to the main processing line, the line may be flushed with buffer to an in-line waste bag. Addition of other components may be performed after zeroing a connected formulation bag on a hanging load cell and peristaltic pumping the desired amount of volume by weight. Protein and buffer solutions may be passed through a closed, disposable sterilizing grade filtration assembly into a sterile bag where additional components (e.g., adjuvant) may be directly added. For vaccines, at this time, a technology does not exist to sterile filter aluminum-based adjuvants due to particle size; however, such filtration is contemplated herein. Adjuvant may be added directly to the formulation intermediates or alternatively, to the final formulation reservoir. This allows adsorption (e.g., may include pre-adsorption) of the antigens onto the aluminum-based adjuvant, a requirement for the processing of some vaccine products. The ingredient lines are connected to the disposable, sterile assemblies using a sterile connection device, such as a tube welder or sterile connector.

A standard sterilizing filtration has four process stages: preparation/flushing, pre-integrity testing, filtration, and post-integrity testing (Baumfalk and Finazzo, 2006). To prevent multiple sterile filter integrity tests for single component filtration, each taking up to 15 minutes per filter, an assembly was designed to sterile filter components in one closed, single-use process. A second filter may be added as a redundant step to satisfy regulatory expectations. In-line bioburden sampling also preferably supports no more than 10 colony-forming units/100 mL of product to be filtered. The system described herein also contains one or more sterilizing filters, having a pore size at least 0.5 μm, and more preferably 0.1-0.45 μm in size. Filters are typically included as well. Filters may be of any suitable pore size, but are typically from about 0.2 to 0.7 μm. Other suitable pore sizes include, for example, about 0.22, 0.45, 0.5, and/or 0.65 μm. Suitable filter materials include but are not limited to, for example, polyvinylidene fluoride (PVDF) and polyethersulphone (PES), or combinations thereof (PVDF/PES). Suitable filters include, for example, Millipore Millipak 20, Sartorius Sartopore 2, Pall EBV, Pall EKV and/or Pall EDF. Where more than one filter is used in the system, the filters may be the same or different according to either brand or pore size. For instance, where two filters are utilized, the first may have a pore size of about 0.2 to 0.7 μm (e.g., about 0.22, 0.45, 0.5, and/or 0.65 μm) and second a pore size of about 0.2 to 0.7 μm (e.g., about 0.22, 0.45, 0.5, and/or 0.65 μm). In certain embodiments, the first filter has a pore size of about 0.22, 0.45, 0.5, and/or 0.65 μm and the second a pore size of about 0.22 μm. A number of filtration studies may be carried out to demonstrate that process outputs fall within expected error ranges or satisfy pre-determined criteria for successful multivalent filtration and formulation. These processes are typically carried out at ambient temperature, although other temperatures may also be utilized. Other suitable filters and filtration systems may also be suitable as would be understood by one of skill in the art.

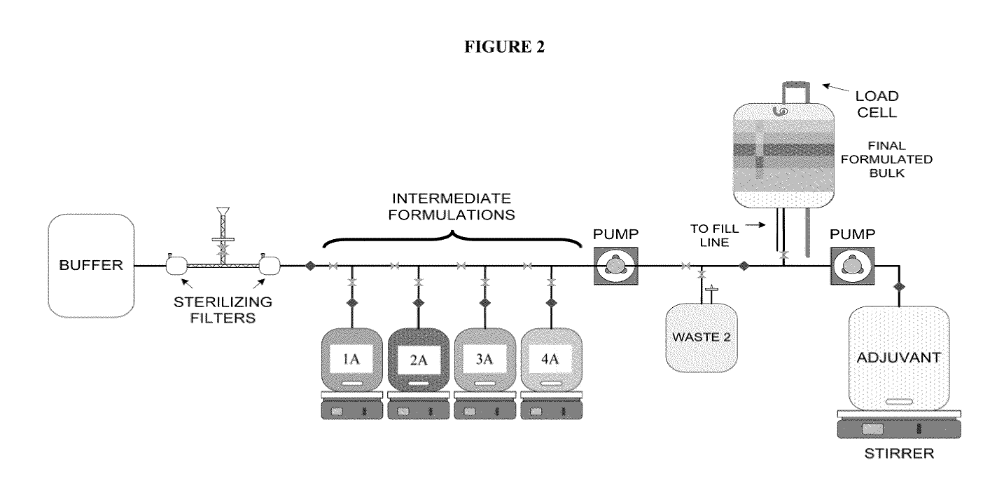

An exemplary sterile, closed, disposable system may include, for example, one or more single-use, pre-sterilized bags, filters, connectors, and/or tubing assemblies as shown in FIG. 1. A preliminary step in using the system may include flushing of the lines and an inline, pre-filter integrity test. FIG. 2 illustrates an exemplary system for blending intermediate formulations into a final formulated bulk bag. Dilution and addition of other components (e.g., adjuvant(s)) may take place at this step to achieve the desired final concentrations. Bulk product may be tube sealed from the line for mixing prior to filling. Individual formulations of a single active agent (e.g., an antigen) may also be diluted from the original bulk concentration and adjuvanted for individual pre-adsorption. Downstream of the final sterility filter, the system may be considered “closed” from the surrounding environment, eliminating the need for ISO Class 5/Grade A clean room or isolator conditions, increasing sterility assurance, and reducing cleaning steps, cost and energy.

The system may involve passing individual or combined components (e.g., active agent(s), buffer(s), and/or surfactant(s)) of the multi-component biopharmaceutical formulation solutions through a closed, disposable sterilizing grade filtration assembly into a sterile bag where one or more additional components (e.g., one or more adjuvants) are directly added to the formulation. Thus, proteins (e.g., antigens) may be individually (optionally) combined with other components (e.g., adjuvant(s)) and diluted in intermediate bags. For vaccines, this allows pre-adsorption of the antigens onto the adjuvant (e.g., an aluminum-based adjuvant) as a formulated intermediate stock of each antigen prior to final blending into the final formulation, and may be referred to as an intermediate stock antigen formulations.

Multiple active agents (e.g., antigens) may be filtered through the same dual filter assembly with buffer flushing through the filter between each protein filtration to remove the residual proteins from the filter for formulation of intermediate individual stock antigen formulations. The intermediate formulations serve three purposes (with respect to vaccines):
pre-adsorption of an individual antigen onto an adjuvant to better control cross-interactions;
dilution from a highly concentrated bulk (up to 60 times higher than the final formulation concentrations), which allows processing a greater volume of lower concentrate in the lines and into the final formulated bulk; and,
ability to store and re-purpose the intermediate stock concentrates for other similar formulations (e.g., bivalent, trivalent, quadrivalent, pentavalent) or doses.

Instead of blending all proteins together prior to filtration, the system described herein allows for controlled, successive protein filtration, preventing potential unwanted interactions at filter face (e.g., binding, clogging). Important parameters involved in filter selection include materials, compatibility, wettability, sterilization, adsorption, structure, and membrane pore size, distribution and thickness (Cardona and Inseal, 2006; Motzkau and Okhio, 2005). In addition, to compare the performance of these filters further, throughput per square meter of the filters can be measured; though one must consider the geometry and effective filtration area to avoid non-linear calculations (Priebe and Jornitz, 2006). The effluent should be tested to ensure minimal protein loss (Cordona and Inseal, 2006). Lower flushing volumes reduce waste and time of processing while still maintaining a high quality of filtrate. Once the desired filtration system has been fully developed, additional performance testing including microbial retention, integrity and extractables/leachables should be initiated (Motzkau and Okhio, 2005). Further adsorption studies are necessary at time of process validation (Motzkau and Okhio, 2005).

In the system described herein, ingredient addition may be based on product specific gravity, desired volumes by weight, and zeroing of bag weight in-line prior to addition. Small bags (1 L) in series, such as those containing intermediates, are prone to moving around on scales or balances, leading to inaccuracies when attempting to measure weight in bags. Accordingly, load cells may be supported by a post and bracketing assembly designed to weigh suspended bags during addition. These may be selected for their ability to withstand measurement disturbances from side loads (bag swaying) and they have moveable load points, making it convenient to hang bags of different configurations. In addition, these should have high individual accuracy (e.g., a combined error of 0.02% and repeatability of 0.01%), and preferably, be designed to discount measurements due to thermal or vibration interference. Bags may be primed, tared, and weighed using the device with a microprocessor-based control with display. Consideration should also be made for flow into intermediate and final formulation bags as they are suspended while ingredients are pumped into these bags. Pumping activities must be properly sequenced with opening and closing of the lines. For small scale manufacturing, this may be accomplished manually. Readings from the load cells once ingredients have been pumped into the hanging bags preferably may have an average percentage difference of ±0.15% (n=35, practical minimum and maximum weights applied) compared with target weight.

In addition to weighing, manufacturing of multi-component biopharmaceutical compositions in disposables can require a variety of processes that occur in parallel or immediately following component addition, including:
Suspension of ingredients prior to and during dispensing;
Blending of intermediates or final formulated bulks after ingredient addition;
Dissolving;
Storage;
Heating/cooling;
Suspension of final formulation prior to and during filling Mechanical attributes to interface these processes should also be considered, including but not limited to:
Type of mixing (e.g., wave, impeller, paddle);
Agitator location, shape, and size relative to vessel, as applicable;
Mixing system parameters (e.g., speed, pitch);
Processing line length and diameter;
Bag and holding vessel size, shape, rigidity, placement;
Bag assembly suitable processing and storage temperature ranges; and,
Bag internal/external accessories (e.g., tubing, baffles, jacketing) and sterilization of product contact mixing components.

During a final blending step or a fill, material may need to stay suspended in the bag while dispensed either into another container or directly to the final filled container (e.g., vial, syringe). In regards to the location of the outlet line, the line should be pumped out of the container during mixing without addition of air in the lines. A clip tube or bottom drain is ideal for preventing air from getting into the lines, otherwise air must be evacuated from the bags. “Scaling up” this system may require a rigid container to hold the bags in the assembly and inverting load cells, as well as larger capacity bags, filters, connectors, tubing, pumps, mixing, weighing and controls to reduce processing steps.

Common quantitative indicators used to measure mixing studies are typically those that test a variable as a function of mixing time (Tm), such as when turbidity, pH, or conductivity reaches steady state or homogeneity at Tm. When mixing systems are engineered around a multivalent product, chemical and physical characteristics (e.g:, foaming-prone excipients, aggregation-prone proteins and heavy mineral-based suspensions), ingredients, stage of manufacturing and target volumes and concentrations must be considered. While the above-mentioned test methods support certain aspects of efficient mixing, for a multivalent formulation, the product and adjuvant concentrations, for vaccines, antigen adsorption to adjuvant and other exchanges should also be tested as a function of Tm or predetermined mixing parameters. Additional studies may be performed to quantify protein loss across the filtration assembly, identification of leachables from disposable assemblies with surfactants and adjuvants, determination if order of component addition induces unwanted aggregation and characterization of process conditions.

A “station” is also typically provided for mixing the formulations contained within the bags with one another to produce a final formulation. This station typically includes the devices needed to combine the formulations together to produce a sterile, homogenous mixture thereof. For instance, the station may comprise a container for the formulation that is compatible with a device or system for mixing or homogenizing the formulation without disrupting the integrity of the active agents contained therein. For instance, the mixing or homogenizing system may include a magnetic stir bar and a stir plate including a source of magnetic energy for rotating the magnetic stir bar that is contained within the bag. Alternatively, a pump may be utilized.

Buffers may be used to maintain the stability and otherwise support the integrity of the components forming the biopharmaceutical formulation. A suitable buffer is any that exerts a desired effect upon the formulation. For instance, a buffer may be used to provide, stabilize, and/or maintain the pH of the formulation. Exemplary buffers that may be used as described herein include but are not limited to, for example, TAPS (3-{[tris(hydroxymethy)methyl]amino}propanesulfonic acid, pH 7.7-9.1), bicine (N,N-bis(2-hydroxyethyl)glycine, pH 7.6-9.0), tris (tris(hydroxymethyl)methylamine, pH 7.5-9.0; e.g, Tris-HCl), HEPES (4-2-hydroxyethyl-1-piperazineethanesulfonic acid, pH 6.8-8.2), TES (2-{[tris(hydroxymethy)methyl]amino}ethanesulfonic acid, pH. 6.8-8.2), MOPS (3-(N-morpholino)propanesulfonic acid, pH 6.5-7.9), PIPES (piperazine-N,N′-bis(2-ethanesulfonic acid), pH 6.1-7.5), cacodylate(dimethylarsinic acid, pH 5.0-7.4), and MES (2-(N-morpholino)ethanesulfonic acid, pH 5.5-6.7), among others. These buffers are typically contained within individual reservoirs of the system but may also be part of the composition comprising a stock solution of active agent. Many other suitable buffers are known to those of skill in the art.

The system described herein also typically contains more than one reservoir containing one or more active agents. Active agents may include any that provide a desired effect (e.g., a therapeutic effect) of the biopharmaceutical formulation upon a host (e.g., human, animal) to whom or to which it is administered. Active agents may be contained within reservoirs alone or in combination with other active agents. Active agents may also be contained within reservoirs with “inactive agents” such as, for example, buffers or other components that are not necessarily active agents. Active agents may include antigens, antibodies, hormones, and/or growth factors, and may be combined with additional components such as adjuvants, any of which may be in purified form, and may be used alone or in combination with one another.

In some embodiments, the antigens may include one or more “immunogens” for inducing or enhancing an immune response that is beneficial to the host. An immunogen may be a moiety (e.g., polypeptide, peptide or nucleic acid) that induces or enhances the immune response of a host to whom or to which the immunogen is administered. An immune response may be induced or enhanced by either increasing or decreasing the frequency, amount, or half-life of a particular immune modulator (e.g, the expression of a cytokine, chemokine, co-stimulatory molecule). This may be directly observed within a host cell or within a nearby cell or tissue (e.g., indirectly). The immune response is typically directed against a target antigen. For example, an immune response may result from expression of an immunogen in a host following administration thereof to the host. The immune response may result in one or more of an effect (e.g., maturation, proliferation, direct- or cross-presentation of antigen, gene expression profile) on cells of either the innate or adaptive immune system. For example, the immune response may involve, effect, or be detected in innate immune cells such as, for example, dendritic cells, monocytes, macrophages, natural killer cells, and/or granulocytes (e.g., neutrophils, basophils or eosinophils). The immune response may also involve, effect, or be detected in adaptive immune cells including, for example, lymphocytes (e.g., T cells and/or B cells). The immune response may be observed by detecting such involvement or effects including, for example, the presence, absence, or altered (e.g., increased or decreased) expression or activity of one or more immunomodulators such as a hormone, cytokine, interleukin (e.g., any of IL-1 through IL-35), interferon (e.g., any of IFN-I (IFN-α, IFN-β, IFN-ε, IFN-κ, IFN-τ, IFN-ζ, IFN-ω), IFN-II (e.g., IFN-γ), IFN-III (IFN-λ1, IFN-λ2, IFN-λ3)), chemokine (e.g., any CC cytokine (e.g., any of CCL1 through CCL28), any CXC chemokine (e.g., any of CXCL1 through CXCL24), Mip1a), any C chemokine (e.g., XCL1, XCL2), any CX3C chemokine (e.g., CX3CL1)), tumor necrosis factor (e.g., TNF-α, TNF-β)), negative regulators (e.g., PD-1, IL-T) and/or any of the cellular components (e.g., kinases, lipases, nucleases, transcription-related factors (e.g., IRF-1, IRF-7, STAT-5, NFKB, STAT3, STAT1, IRF-10), and/or cell surface markers suppressed or induced by such immunomodulators) involved in the expression of such immunomodulators. The presence, absence or altered expression may be detected within cells of interest or near those cells (e.g., within a cell culture supernatant, nearby cell or tissue in vitro or in vivo, and/or in blood or plasma). Administration of the immunogen may induce (e.g., stimulate a de novo or previously undetected response), enhance and/or suppress an existing response against the immunogen by, for example, causing an increased antibody response (e.g., amount of antibody, increased affinity/avidity) or an increased cellular response (e.g., increased number of activated T cells, increased affinity/avidity of T cell receptors). In certain embodiments, the immune response may be protective, meaning that the immune response may be capable of preventing initiation or continued infection of or growth within a host and/or by eliminating an agent (e.g., a causative agent, such as HIV) from the host.

The formulations described herein may include one or more immunogen(s) from a single source or multiple sources. For instance, immunogens may also be derived from or direct an immune response against one or more viruses (e.g., viral target antigen(s)) including, for example, a dsDNA virus (e.g. adenovirus, herpesvirus, epstein-barr virus, herpes simplex type 1, herpes simplex type 2, human herpes virus simplex type 8, human cytomegalovirus, varicella-zoster virus, poxvirus); ssDNA virus (e.g., parvovirus, papillomavirus (e.g., E1, E2, E3, E4, E5, E6, E7, E8, BPV1, BPV2, BPV3, BPV4, BPV5 and BPV6 (In Papillomavirus and Human Cancer, edited by H. Pfister (CRC Press, Inc. 1990); Lancaster et al., Cancer Metast. Rev. pp. 6653-6664 (1987); Pfister, et al. Adv. Cancer Res 48, 113-147 (1987)); dsRNA viruses (e.g., reovirus); (+)ssRNA viruses (e.g., picornavirus, coxsackie virus, hepatitis A virus, poliovirus, togavirus, rubella virus, flavivirus, hepatitis C virus, yellow fever virus, dengue virus, west Nile virus); (−)ssRNA viruses (e.g., orthomyxovirus, influenza virus, rhabdovirus, paramyxovirus, measles virus, mumps virus, parainfluenza virus, respiratory syncytial virus, rhabdovirus, rabies virus); ssRNA-RT viruses (e.g. retrovirus, human immunodeficiency virus (HIV)); and, dsDNA-RT viruses (e.g. hepadnavirus, hepatitis B). Immunogens may also be derived from other viruses not listed above but available to one of skill in the art.

With respect to HIV, immunogens may be selected from any HIV isolate. As is well-known in the art, HIV isolates are now classified into discrete genetic subtypes. HIV-1 is known to comprise at least ten subtypes (A, B, C, D, E, F, G, H, J and K). HIV-2 is known to include at least five subtypes (A, B, C, D, and E). Subtype B has been associated with the HIV epidemic in homosexual men and intravenous drug users worldwide. Most HIV-1 immunogens, laboratory adapted isolates, reagents and mapped epitopes belong to subtype B. In sub-Saharan Africa, India and China, areas where the incidence of new HIV infections is high, HIV-1 subtype B accounts for only a small minority of infections, and subtype HIV-1 C appears to be the most common infecting subtype. Thus, in certain embodiments, it may be preferable to select immunogens from HIV-1 subtypes B and/or C. It may be desirable to include immunogens from multiple HIV subtypes (e.g., HIV-1 subtypes B and C, HIV-2 subtypes A and B, or a combination of HIV-1 and HIV-2 subtypes) in a single immunological formulation. Suitable HIV immunogens include ENV, GAG, POL, NEF, as well as variants, derivatives, and fusion proteins thereof, for example. Any of these may be encoded by a polynucleotide within a recombinant vector, and/or used in combination with a recombinant vector as part of an immunization strategy.

Immunogens may also be derived from or direct an immune response against one or more bacterial species (spp.) (e.g., bacterial target antigen(s)) including, for example, Bacillus spp. (e.g., Bacillus anthracis), Bordetella spp. (e.g., Bordetella pertussis), Borrelia spp. (e.g., Borrelia burgdorferi), Brucella spp. (e.g., Brucella abortus, Brucella canis, Brucella melitensis, Brucella suis), Campylobacter spp. (e.g., Campylobacter jejuni), Chlamydia spp. (e.g., Chlamydia pneumoniae, Chlamydia psittaci, Chlamydia trachomatis), Clostridium spp. (e.g., Clostridium botulinum, Clostridium difficile, Clostridium perfringens, Clostridium tetani), Corynebacterium spp. (e.g., Corynebacterium diptheriae), Enterococcus spp. Enterococcus faecalis, enterococcus faecum), Escherichia spp. Escherichia coli), Francisella spp. (e.g., Francisella tularensis), Haemophilus spp. (e.g., Haemophilus influenza), Helicobacter spp. (e.g., Helicobacter pylori), Legionella spp. (e.g., Legionella pneumophila), Leptospira spp. (e.g., Leptospira interrogans), Listeria spp. (e.g., Listeria monocytogenes), Mycobacterium spp. (e.g., Mycobacterium leprae, Mycobacterium tuberculosis), Mycoplasma spp. (e.g., Mycoplasma pneumoniae), Neisseria spp. (e.g., Neisseria gonorrhea, Neisseria meningitidis), Pseudomonas spp. (e.g., Pseudomonas aeruginosa), Rickettsia spp. (e.g., Rickettsia rickettsii), Salmonella spp. (e.g., Salmonella typhi, Salmonella typhinurium), Shigella spp. (e.g., Shigella sonnei), Staphylococcus spp. (e.g., Staphylococcus aureus, Staphylococcus epidermidis, Staphylococcus saprophyticus, coagulase negative staphylococcus (e.g., U.S. Pat. No. 7,473,762)), Streptococcus spp. (e.g., Streptococcus agalactiae, Streptococcus pneumoniae, Streptococcus pyrogenes), Treponema spp. (e.g., Treponema pallidum), Vibrio spp. Vibrio cholerae), and Yersinia spp. (Yersinia pestis). Exemplary antigens may include, for example, PhtE (also “protein E”), PcpA (“protein A”), LytB (“protein B”), PhtD (“protein D”), and Pneumolysin (for example, detoxified Ply proteins, such as PlyD1 or PdB (“protein C”) (see, e.g., Examples 2, 4 and 10 herein). Immunogens may also be derived from or direct the immune response against other bacterial species not listed above but available to one of skill in the art.

Immunogens may also be derived from or direct an immune response against one one or more fungal species (spp.) may be detected such as, for example, Actinomyces spp. (e.g., A. israelii, A. bovis, A. naeslundii), Allescheria spp. (e.g., A. boydii), Aspergillus spp. (e.g., A. fumigatus, A. nidulans), Blastomyces spp. (e.g., B. dermatidis), Candida spp. (e.g., C. albicans), Cladosporium spp. (e.g., C. carrionii), Coccidioides spp. (e.g., C. immitis), Cryptococcus spp. (e.g., C. neoformans), Fonsecaea spp. (e.g., F. pedrosoi, F. compacta, F. dermatidis), Histoplasma spp. (e.g., H. capsulatum), Nocardia spp. (e.g., N. asteroids, N. brasiliensis), Keratinomyces spp. (e.g., K. ajelloi), Madurella spp. (e.g., M. grisea, M. mycetomi), Microsporum spp. (e.g., M. adnouini, M. gypseum, M. canis), Mucor spp. (e.g., M. corymbifer, Absidia corymbifera), Paracoccidioides spp. (e.g., P. brasiliensis), Phialosphora spp. (e.g., P. jeansilmei, P. verrucosa), Rhizopus spp. (e.g., R. oryzae, R. arrhizus, R. nigricans), Sporotrichum spp. (e.g., S. Schenkii), and Trichophyton spp. (e.g., T. mentaarophytes, T. rubrum). Immunogens may also be derived from other fungal species not listed above as would be understood by one of skill in the art.

Immunogens may also be derived from or direct an immune response against one or more parasitic organisms (spp.) (e.g., parasite target antigen(s)) including, for example, Ancylostoma spp. (e.g., A. duodenale), Anisakis spp., Ascaris lumbricoides, Balantidium coli, Cestoda spp., Cimicidae spp., Clonorchis sinensis, Dicrocoelium dendriticum, Dicrocoelium hospes, Diphyllobothrium latum, Dracunculus spp., Echinococcus spp. (e.g., E. granulosus, E. multilocularis), Entamoeba histolytica, Enterobius vermicularis, Fasciola spp. (e.g., F. hepatica, F. magna, F. gigantica, F. jacksoni), Fasciolopsis buski, Giardia spp. (Giardia lamblia), Gnathostoma spp:, Hymenolepis spp. (e.g., H. nana, H. diminuta), Leishmania spp., Loa loa, Metorchis spp. (M. conjunctus, M. albidus), Necator americanus, Oestroidea spp. (e.g., botfly), Onchocercidae spp., Opisthorchis spp. (e.g., O. viverrini, O. felineus, O. guayaquilensis, and O. noverca), Plasmodium spp. (e.g., P. falciparum), Protofasciola robusta, Parafasciolopsis fasciomorphae, Paragonimus westermani, Schistosoma spp. (e.g., S. mansoni, S. japonicum, S. mekongi, S. haematobium), Spirometra erinaceieuropaei, Strongyloides stercoralis, Taenia spp. (e.g., T. saginata, T. solium), Toxocara spp. (e.g., T. canis, T. cati), Toxoplasma spp. (e.g., T. gondii), Trichobilharzia regenti, Trichinella spiralis, Trichuris trichiura, Trombiculidae spp., Trypanosoma spp., Tunga penetrans, and/or Wuchereria bancrofti. Immunogens may also be derived from or direct the immune response against other parasitic organisms not listed above but available to one of skill in the art.

immunogens may be derived from or direct the immune response against tumor target antigens (e.g., tumor target antigens). The term tumor target antigen (TA) may include both tumor-associated antigens (TAAs) and tumor-specific antigens (TSAs), where a cancerous cell is the source of the antigen. A TA may be an antigen that is expressed on the surface of a tumor cell in higher amounts than is observed on normal cells or an antigen that is expressed on normal cells during fetal development. A TSA is typically an antigen that is unique to tumor cells and is not expressed on normal cells. TAs are typically classified into five categories according to their expression pattern, function, or genetic origin: cancer-testis (CT) antigens (e.g., MAGE, NY-ESO-1); melanocyte differentiation antigens (e.g., Melan A/MART-1, tyrosinase, gp100); mutational antigens (e.g., MUM-1, p53, CDK-4); overexpressed ‘self’ antigens (e.g., HER-2/neu, p53); and, viral antigens (e.g., HPV, EBV). Suitable TAs include, for example, gp100 (Cox et al., Science, 264:716-719 (1994)), MART-1/Melan A (Kawakami et al., J. Exp. Med., 180:347-352 (1994)), gp75 (TRP-1) (Wang et al., J. Exp. Med., 186:1131-1140 (1996)), tyrosinase (Wolfe) et al., Eur. J. Immunol., 24:759-764 (1994)), NY-ESO-1 (WO 98/14464; WO 99/18206), melanoma proteoglycan (Hellstrom et al., J. Immunol., 130:1467-1472 (1983)), MAGE family antigens (e.g., MAGE-1, 2,3,4,6, and 12; Van der Bruggen et al., Science, 254:1643-1647 (1991); U.S. Pat. No. 6,235,525), BAGE family antigens (Boel et al., Immunity, 2:167-175 (1995)), GAGE family antigens (e.g., GAGE-1,2; Van den Eynde et al., J. Exp. Med., 182:689-698 (1995); U.S. Pat. No. 6,013,765), RAGE family antigens (e.g., RAGE-1; Gaugler et al., Immunogenetics, 44:323-330 (1996); U.S. Pat. No. 5,939,526), N-acetylglucosaminyltransferase-V (Guilloux et at., J. Exp. Ivied:, 183:1173-1183 (1996)), p15 (Robbins et al., J. Immunol. 154:5944-5950 (1995)), β-catenin (Robbins et al., J. Exp. Med., 183:1185-1192 (1996)), MUM-1 (Coulie et al., Proc. Natl. Acad-Sci. USA, 92:7976-7980 (1995)), cyclin dependent kinase-4 (CDK4) (Wolfe) et al., Science, 269:1281-1284 (1995)), p21-ras (Fossum et at., Int. J. Cancer, 56:40-45 (1994)), BCR-ab1 (Bocchia et al., Blood, 85:2680-2684 (1995)), ₚ53 (Theobald et al., Proc. Natl. Acad. Sci. USA, 92:11993-11997 (1995)), p185 HER2/neu (erb-B1; Fisk et al., J. Exp. Med., 181:2109-2117 (1995)), epidermal growth factor receptor (EGFR) (Harris et al., Breast Cancer Res. Treat, 29:1-2 (1994)), carcinoembryonic antigens (CEA) (Kwong et al., J. Natl. Cancer Inst., 85:982-990 (1995) U.S. Pat. Nos. 5,756,103; 5,274,087; 5,571,710; 6,071,716; 5,698,530; 6,045,802; EP 263933; EP 346710; and, EP 784483); carcinoma-associated mutated mucins MUC-1 gene products; Jerome et al., J. Immunol:, 151:1654-1662 (1993)); EBNA gene products of EBV (e.g., EBNA-1; Rickinson et al., Cancer Surveys, 13:53-80 (1992)); E7, E6 proteins of human papillomavirus (Ressing et al., J. Immunol, 154:5934-5943 (1995)); prostate specific antigen (PSA; Xue et al., The Prostate, 30:73-78 (1997)); prostate specific membrane antigen (PSMA; Israeli, et al., Cancer Res., 54:1807-1811 (1994)); idiotypic epitopes or antigens, for example, immunoglobulin idiotypes or T cell receptor idiotypes (Chen et al., J. Immunol., 153:4775-4787 (1994)); KSA (U.S. Pat. No. 5,348,887), kinesin 2 (Dietz, et al. Biochem Biophys Res Commun 2000 Sep. 7; 275(3):731-8), HIP-55, TGFβ-1 anti-apoptotic factor (Toomey, et al. Br J Biomed Sci 2001;58(3):177-83), tumor protein D52 (Bryne J. A., et al., Genomics, 35:523-532 (1996)), HIFT, NY-BR-1 (WO 01/47959), NY-BR-62, NY-BR-75, NY-BR-85, NY-BR-87 and NY-BR-96 (Scanlan, M. Serologic and Bioinformatic Approaches to the Identification of Human Tumor Antigens, in Cancer Vaccines 2000, Cancer Research Institute, New York, N.Y.), and/or pancreatic cancer antigens (e.g., SEQ ID NOS: 1-288 of U.S. Pat. No. 7,473,531). Immunogens may also be derived from or direct the immune response against include TAs not listed above but available to one of skill in the art.

Vaccines suitable for preparation using the systems described herein are typically “multivalent”. A multivalent vaccine is an antigenic preparation including more than one infectious agent or several different antigenic determinants of a single agent. For example, described herein are multivalent vaccines containing at least, for example, two, three, four five or more different recombinant proteins formulated as a combination vaccine. The system described herein is suitable for the development of biopharmaceutical compositions that may be used anywhere from concept through Phase III clinical testing and beyond (e.g., Phase IV, commercialization). For instance, Phase I/II typically requires less than 200 doses for a trial, while a two to five liter (2-5 L) final formulation bulk size is needed.

Active agents may also be antibodies. The term “antibody” or “antibodies” includes whole or fragmented antibodies in unpurified or partially purified form (i.e., hybridoma supernatant, ascites, polyclonal antisera) or in purified form. A “purified” antibody is one that is separated from at least about 50% of the proteins with which it is initially found (i.e., as part of a hybridoma supernatant or ascites preparation). Preferably, a purified antibody is separated from at least about 60%, 75%, 90%, or 95% of the proteins with which it is initially found. Suitable derivatives may include fragments (i.e., Fab, Fab₂ or single chain antibodies (Fv for example)), as are known in the art. The antibodies may be of any suitable origin or form including, for example, murine (i.e., produced by murine hybridoma cells), or expressed as humanized antibodies, chimeric antibodies, human antibodies, and the like. Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Harlow, et al. Using Antibodies: A Laboratory Manual, Portable Protocol No. 1, 1998; Kohler and Milstein, Nature, 256:495 (1975)); Jones et al. Nature, 321:522-525 (1986); Riechmann et al. Nature, 332:323-329 (1988); Presta (Curr. Op. Struct. Biol., 2:593-596 (1992); Verhoeyen et al. (Science, 239:1534-1536 (1988); Hoogenboom et al., J. Mol. Biol., 227:381 (1991); Marks et al., J. Mol. Biol., 222:581 (1991); Cole et al., Monoclonal Antibodies and Cancer Therapy, Alan R. Liss, p. 77 (1985); Boerner et al., J. Immunol., 147(1):86-95 (1991); Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); as well as U.S. Pat. Nos. 4,816,567; 5,545,807; 5,545,806; 5,569,825; 5,625,126; 5,633,425; and, 5,661,016). In certain applications, the antibodies may be contained within hybridoma supernatant or ascites and utilized either directly as such or following concentration using standard techniques. In other applications_{;} the antibodies may be further purified using, for example, salt fractionation and ion exchange chromatography, or affinity chromatography using Protein A, Protein G, Protein A/G, and/or Protein L ligands covalently coupled to a solid support such as agarose beads, or combinations of these techniques. The antibodies may be stored in any suitable format, including as a frozen preparation (i.e., −20° C. or −70° C.), in lyophilized form, or under normal refrigeration conditions (i.e., 4° C.). When stored in liquid form, it is preferred that a suitable buffer such as Tris-buffered saline (TBS) or phosphate buffered saline (PBS) is utilized. The antibodies described herein may be prepared as injectable preparations, such as in suspension in a non-toxic parenterally acceptable diluent or solvent. Suitable vehicles and solvents that may be utilized include water, Ringer's solution, and isotonic sodium chloride solution, TBS and PBS, among others. It is preferred that the antibodies be suitable for use in vivo.

Suitable hormones include but are not hinted to antidiuretic hormone, proopiomelanocortin, luteinizing hormone, follicle stimulating hormone, adrenocorticotrophic hormone, growth hormone, prolactin, melanocyte stimulating hormone, thyroid stimulating hormone, insulin, triiodothyronine, thyroxine, cortisol, dehydroepiandrostendione, an estrogen (e.g., estradiol, estrone, estriol), progesterone, testosterone, dihydrotestosterone, inhibin, progesterone, and estriol, for example. Suitable, exemplary growth factors include but are not limited to bone morphogenic proteins (BMPs), epidermal growth factor (EGF), erythropoietin (EPO), fibroblast growth factor (FGF), granulocyte-colony stimulating factor (G-CSF), granulocyte-macrophage colony stimulating factor (GM-CSF), growth differentiation factor-9 (GDF9), hepatocyte growth factor (HGF), insulin-like growth factor (IGF), myostatin (GDF-8), neurotrophins (e.g., nerve growth factor (NGF)), platelet-derived growth factor (PDGF), thrombopoietin (TPO), transforming growth factor alpha (TGF-α), transforming growth factor beta (TGF-β), and vascular endothelial growth factor (VEGF), among others.

As described above, in certain embodiments, one or more additional components may also be added to form a final formulation. In some embodiments, the active agent may be an antigen and/or the one or more additional components may be one or more adjuvants. An immunogen may also be administered in combination with one or more adjuvants to boost the immune response. Adjuvants may also be included to stimulate or enhance the immune response. Non-limiting examples of suitable adjuvants include those of the gel-type (e.g., aluminum hydroxide/phosphate (“alum adjuvants”), calcium phosphate), of microbial origin (muramyl dipeptide (MDP)), bacterial exotoxins (cholera toxin (CT), native cholera toxin subunit B (CTB), E. coli labile toxin (LT), pertussis toxin (PT), CpG oligonucleotides, BCG sequences, tetanus toxoid, monophosphoryl lipid A (MPL) of, for example, E. coli, Salmonella minnesota, Salmonella typhimurium, or Shigella exseri), particulate adjuvants (biodegradable, polymer microspheres), immunostimulatory complexes (ISCOMs)), oil-emulsion and surfactant-based adjuvants (Freund's incomplete adjuvant (FIA), microfluidized emulsions (MF59, SAF), saponins (QS-21)), synthetic (muramyl peptide derivatives (murabutide, threony-MDP), nonionic block copolymers (L121), polyphosphazene (PCCP), synthetic polynucleotides (poly A:U, poly I:C), thalidomide derivatives (CC-4407/ACTIMID)), RH3-ligand, or polylactide glycolide (PLGA) microspheres, among others. Fragments, homologs, derivatives, and fusions to any of these toxins are also suitable, provided that they retain adjuvant activity. Suitable mutants or variants of adjuvants are described, e.g., in WO 95/17211 (Arg-7-Lys CT mutant), WO 96/6627 (Arg-192-Gly LT mutant), and WO 95/34323 (Arg-9-Lys and Glu-129-Gly PT mutant). Additional LT mutants that can be used in the methods and compositions of the invention include, e. g., Ser-63-Lys, Ala-69-Gly,Glu-110-Asp, and Glu-112-Asp mutants. Other suitable adjuvants are also well-known in the art.

As an example, metallic salt adjuvants such alum adjuvants are well-known in the art as providing a safe excipient with adjuvant activity. The mechanism of action of these adjuvants are thought to include the formation of an antigen depot such that antigen may stay at the site of injection for up to 3 weeks after administration, and also the formation of antigen/metallic salt complexes which are more easily taken up by antigen presenting cells. In addition to aluminum, other metallic salts have been used to adsorb antigens, including salts of zinc, calcium, cerium, chromium, iron, and berilium. The hydroxide and phosphate salts of aluminum are the most common. Formulations or compositions containing aluminum salts, antigen, and an additional immunostimulant are known in the art. An example of an immunostimulant is 3-de-O-acylated monophosphoryl lipid A (3D-MPL).

One or more cytokines and/or chemokines may also be suitable adjuvants (Parmiani, et al. Immunol Lett 2000 Sep. 15; 74(1): 41-4; Berzofsky, et al. Nature Immunol. 1: 209-219). Suitable cytokines include, for example, interleukin-2 (IL-2) (Rosenberg, et al. Nature Med. 4: 321-327 (1998)), IL-4, IL-7, IL-12 (reviewed by Pardoll, 1992; Harries, et al. J. Gene Med. 2000 July-August; 2(4):243-9; Rao, et al. J. Immunol. 156: 3357-3365 (1996)), IL-15 (Xin, et al. Vaccine, 17:858-866, 1999), IL-16 (Cruikshank, et al. J. Leuk Biol. 67(6): 757-66, 2000), IL-18 (J. Cancer Res. Clin. Oncol. 2001. 127(12): 718-726), GM-CSF (CSF (Disis, et al. Blood, 88: 202-210 (1996)), tumor necrosis factor-alpha (TNF-α), or interferon-gamma (INF-γ). Chemokines may also be utilized. For example, fusion proteins comprising CXCL10 (IP-10) and CCL7 (MCP-3) fused to a tumor self-antigen have been shown to induce anti-tumor immunity (Biragyn, et al. Nature Biotech. 1999, 17: 253-258). The chemokines CCL3 (MIP-1α) and CCL5 (RANTES) (Boyer, et al. Vaccine, 1999, 17 (Supp. 2): S53-S64) may also be of use in practicing the present invention. Other suitable cytokines and chemokines are known in the art.

Formulations produced as described herein may be prepared as pharmaceutical compositions. The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” may be a composition comprising a therapeutically effective amount of an active agent contained within a formulation. The terms “effective amount” and “therapeutically effective amount” each refer to the amount of active agent required to observe the desired therapeutic effect (e.g., induce or enhance and immune response).

Injectable preparations, such as sterile injectable aqueous or oleaginous suspensions, may be formulated according to known methods using suitable dispersing or wetting agents and suspending agents. The injectable preparation may also be a sterile injectable solution or suspension in a non-toxic parenterally acceptable diluent or solvent. Suitable vehicles and solvents that may be employed are water, Ringer's solution, and isotonic sodium chloride solution, among others. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

Pharmaceutical compositions may take any of several forms and may be administered by any of several routes. The compositions may be administered via a parenteral route (intradermal, intramuscular or subcutaneous) to induce an immune response in the host. Alternatively, the composition may be administered directly into a lymph node (intranodal) or tumor mass (e.g., intratumoral administration). Preferred embodiments of administratable compositions include, for example, one or more active agents in liquid preparations such as suspensions, syrups, or elixirs. Preferred injectable preparations include, for example, nucleic acids or polypeptides suitable for parental, subcutaneous, intradermal, intramuscular or intravenous administration such as sterile suspensions or emulsions. For example, active agents may be prepared in admixture with a suitable carrier, diluent, or excipient such as sterile water, physiological saline, glucose or the like. The composition may also be provided in lyophilized form for reconstituting, for instance, in isotonic aqueous, saline buffer. In addition, the compositions can be co-administered or sequentially administered with one another, other antiviral compounds, other anti-cancer compounds and/or compounds that reduce or alleviate ill effects of such agents.

As previously mentioned, while the compositions described herein may be administered as the sole active pharmaceutical agent, they can also be used in combination with one or more other compositions or agents (e.g., other immunogens, co-stimulatory molecules, adjuvants). When administered as a combination, the individual components can be formulated as separate compositions administered at the same time or different times, or the components can be combined as a single composition. In one embodiment, a method of administering to a host a first form of an immunogen and subsequently administering a second form of the immunogen, wherein the first and second forms are different, and wherein administration of the first form prior to administration of the second form enhances the immune response resulting from administration of the second form relative to administration of the second form alone, is provided. Also provided are compositions for administration to the host. For example, a two-part immunological composition where the first part of the composition comprises a first form of an immunogen and the second part comprises a second form of the immunogen, wherein the first and second parts are administered separately from one another such that administration of the first form enhances the immune response against the second form relative to administration of the second form alone, is provided. The immunogens, which may be the same or different, are preferably derived from the infectious agent or other source of immunogens. The multiple immunogens may be administered together or separately, as a single or multiple compositions, or in single or multiple recombinant vectors.

A kit is also provided which may include a system comprising a buffer reservoir, multiple reservoirs of active agents, each reservoir containing a different active agent or combination of active agents, one or more pumps, one or more sterilizing filters, multiple single-use, pre-sterilized bags, each bag containing a formulation of an active agent or combination of active agents corresponding to those in the reservoirs, a station for mixing the formulations contained within the bags with one another, which optionally contain one or more additional components (e.g., an adjuvant) to form a final formulation. The kit may also include some or all of these components such as, for example, one or more buffer reservoirs, one or more reservoirs of active agents, one or more sterilizing filters, one or more bags containing a formulation of active agent and/or one or more additional components (e.g., adjuvant). These components may be adapted for use in a system comprising one or more pumps. Additionally, the kit can include instructions for using these components to prepare the formulations described herein.

Certain embodiments are further described in the following examples. These embodiments are provided as examples only and are not intended to limit the scope of the claims in any way.

### EXAMPLES

### Example 1

### Materials and Methods

Equipment used in a sterile, closed, disposable system cannot be intrusive, meaning, no part of the equipment can come in direct contact with the product, unless this part is also sterile, and such that it maintains the sterility and closed system of the overall assembly. Also due to small-scale processing, each piece of equipment and device is small and portable so that the system can be transferred easily from lab bench scale to a cleanroom without doubling capital.

The Wave 20/50EH Electric WaveMixer with Touchpanel (GE Healthcare Life Sciences) is an electrical rocker where bags are placed in a SS holder that fits on a base and unit provides mixing with heater and temperature control for thawing, warming and mixing applications. The Wave concept of non-invasive mixing provides low fluid velocity to reduce shear forces and protect products from damage and foaming. Agitation is achieved using gravity to accelerate the fluid contained in the bag. The wave sweeps up solids and disperses them into the liquid. Direction reversals cause a reciprocating chaotic motion (Source: Singh, 2000). This unit is used for mixing of bulk ingredients, in-process and final formulations in bags.

The BLH/Vishay Kis 3 Shear Beam Load Cell (Vishay BLH) with support post and bracketing assembly was used to weigh suspended bags during the dispensing of formulation ingredients. The load cell works similarly to a scale, however, it measures strain based on shear and is more accurate and precise. There is limited interference from nearby assemblies as the bags are suspended and tubing secured using weighted tubing holders. Bags were primed, tared and weighed using the device with a microprocessor-based control and panel readout. The accuracy of these units is 0.02%, and there are no effects of reading by thermal or vibration interference, and the device has moveable load points. The device also withstands both high lateral forces and have a wide temperature range of −40 to +80° C.

The Sartochek Filter Integrity Tester unit (Sartorius Stedim Biotech S.A.) is an automatic standard, microprocessor-controlled filter integrity tester to test the integrity of vent and liquid membrane filters. It is used for its bubble point testing to test integrity of the filters from the multivalent disposable formulation system.

A peristaltic pump was used to non-invasively and gently pump and dispense liquids from one container or system to another. This provides more control for fluid movement in the disposable formulation system.

The Wave Biotech Hot Lips Tube Sealer (Wave Europe Pvt. Ltd) was used to seal the outside of the tubing while the inside remains sterile preventing leakage or contact with foreign materials and equipment. It can be used with liquid-filled thermoplastic tubing such as C-Flex.

The Disposable Bag Assemblies (TC-TECH) (Thermo Fisher Scientific) assemblies consisting of bags, tubing, connectors and filters were custom-designed specifically for the purposes of the multivalent formulation. They are designed by the end-user and bag manufacturer (formerly Sartorius/Stericon, now Thermofisher), assembled, sealed in bags and then gamma irradiated by a validated process.

Bags: TC-TECH/Thermofisher, film AF-793 with a ULDPE main product contact layer. Bags used in the system range from 60 mL to 5 L. The IL and 5 L bags contain a 2×⅜″ Teflon-coated stir bar (component numbers CX22782S and SV20887.01) intended for mixing on a stir plate.

C-Flex Tubine: Opaque TPE tubing is heat sealable and weldable. Low protein binding minimizes potential for active ingredient loss. Tubing is fully characterized in accordance with USP 24 guidelines. Formulation 072, Shore A, 60. Formulation 050, Shore A, 50.

Sartorius Sartopore 2 filters (Sartorius Stedim): These filters are gamma irradiated in a full assembly provided by Thermofisher prior to use. The filters consist of a 0.45 μm asymmetric polyethersulphone (PES) filter followed by a 0.2 μm asymmetric PES end filter, and exhibit broad chemical compatibility of pH 1 through 14.

The MGA Technologies Tube Welder is a sterile, connecting device that was developed by MGA Technologies to improve sterility assurance during aseptic connections between pieces of C-Flex tubing. The device operates by using a heated Teflon blade (215° C.) that cuts tubing. While hot and in contact with the blade the tubing ends to be connected are aligned and pressed together. Tubing can be dry or moist (but not liquid-filled) for the operation. As the tubing cools a sterile weld is formed and during the process the internal bore of the tubing is never exposed to the external atmosphere. The connection is performed without open aseptic manipulation.

The following is a list of calibration and validation that may apply for equipment used for the formulation process (Table 1).

TABLE 1
Disposables Equipment Calibration and Validation Requirements

| Equipment | Calibration | Validation (required for GMP) |
|---|---|---|
| Peristaltic pump | NO | NO |
| Tube welder | YES | IOQ, PQ |
| Tube sealer | YES | IOQ, PQ through broth |
| Load cells | YES | NO |
| Magnetic stirrer | NO | NO |
| Filter Integrity Tester | YES | IOQ |

During this process, the formulator must obtain formulation ingredients in closed containers with weldable tubing, with sterility and/or bioburden, specific gravity and concentration test records (as applicable); ensure all equipment is fully operational with maintenance/use logbooks in place, calibrated, validated (where required) and setup at the desired area for formulation; assemble the configuration (e.g., tube welding one bag to another); relocate the mobile equipment during different parts of the formulation; and, filter integrity testing, pre- and post-filtration; observe the load cell control panels during pump dispensing to ensure the dispensing weight meets calculated target; observe the lines for air bubbles and ingredients at certain times in the process; clamp the lines with haemostats; and, execute and populate the batch-specific procedural documents.

Calculations for a multivalent formulation can be complicated, especially when an intermediate formulation and several ingredients are required (e.g. excipients). It is convenient to setup the calculations in a spreadsheet that has entry cells for “knowns”, variables (shown in the bold squares) and with formulas for calculations for outputs. Formulation volumes are back-calculated based on the number of filled vials required for the study. In some cases, there is only so much material (e.g. protein) to work with, so this can be a limiting factor. Volumes to dispense are based on weight by way of the known specific gravity of the ingredients.

As described herein, studies have been performed to develop the formulation process in the disposables system, testing antigen concentration and aluminum content as major outcomes. One study compared two different processing scenarios for a quadrivalent formulation. Another study optimized production of bivalent and trivalent formulations.

### Example 2

### Study CA-08-162A

The single-use assemblies used in this Example consisted of two (2) and three (3) D bags connected to a manifold of tubing, connectors, and filters. These were custom-made by the bag manufacturer, assembled, sealed into bags, and gamma-irradiated using a validated sterilization method. Selected primarily for their inert compatibility properties, gamma-irradiation stability, quality testing, biological safety testing, and low leachables/extractables profile (Cardona and Allen, 2006), the film and tubing remained constant throughout these experiments. The bags, tubing and filters were supported by stands and holding apparatuses assuring proper alignment and dispensing control for the connections.

The process was designed for a vaccine formulation comprising proteins, adjuvants and excipients in the final product. The stages encompassed include filtration, intermediate formulation, final formulation, and blending at the bulk product stage (FIGS. 1-4).

A number of filtration studies were carried out to demonstrate that process outputs fall within expected error ranges or satisfy pre-determined criteria for successful multivalent filtration and formulation. All experimental processes were performed at ambient temperature. On the recommendation of leading filter manufacturers for filtration of proteins, each of Filter M, Filter E and Filter S were selected based on four critical specifications: 1) an appropriate surface area for the volumes required; 2) membrane types and construction suitable for sterile filtration of recombinant proteins (up to 100 000 Daltons) and buffers; 3) filter membrane materials are designed for low binding of proteins; 4) filters are hydrophilic, wettable without use of wetting agent and can be gamma-irradiated (Cardona and Inseal, 2006). Filter M has a polyvinylidene fluoride (PVDF) membrane, Filter E has both PVDF and polyethersulphone (PES) membranes, and filter S membrane is PES (Table 2).

TABLE 2
Sterile Filter Technical Information

| Sterile | | | | Filtration |
|---|---|---|---|---|
| Filter | Membrane | Support | Configuration | Area |
| “M” | PVDF | Polycarbonate | 0.22 μm | 100 cm² |
| | | | Stacked disk | |
| | | | filter | |
| “S” | PES | Polypropylene | 0.45 μm + | 150 cm² |
| | | | 0.2 μm | |
| | | | Pleated, | |
| | | | asymmetric | |
| | | | capsule | |
| “E” | PVDF + | Polypropylene | 0.45 μm + | 200 + |
| | PES | | 0.22 μm | 220 cm² |
| | | | Pleated | |
| | | | capsule | |

Physical studies compared the rate of filtration and pressure change of the three sterilizing grade filters (E, M, and S) after filtration in one embodiment of the present invention of up to five antigens (proteins E (antigen phtE), A (antigen PcpA), B (antigen LytB), and D (antigen PhtD)) consecutively and in random order. These antigens can be isolated from the native organism or recombinantly produced. In this embodiment these antigens were recombinantly produced from cloned genes from a Streptococcus pneumonia bacterium. Constant pump speed at infeed was applied. An increase in pressure at the filter could indicate pore clogging. This effect is likely caused by aggregation of the proteins at the filter membrane (Sharma et al., 2008). Comparing the filtration rate of multiple proteins through the smaller disc version of the membrane did not have the same results as the capsule or stacked disk system. For this reason, and to verify the actual filtration assembly system, further experiments were done with the scalable dual in-line capsule/stacked disk filters and filter testing assembly to represent actual filter size, geometry, type of symmetry, volumes, and setup used. Measuring the effluent should be tested to ensure minimal protein loss, (Cordona and Inseal, 2006). Lower flushing volumes reduce waste and time of processing while still maintaining a high quality of filtrate. A second study was performed to measure the volume of buffer necessary to flush the filters to prevent cross-contamination of the protein intermediates prior to final blending. Finally, with the best filtration system selected, percentage protein loss was tested.

The three filters did not clog during antigen filtration and there was no pressure increase, therefore all three filters can be used for the filtration of the antigens tested. There was no direct correlation between order of the proteins added and filtration rate. However, based on the design, for a protein that requires different ingredients (e.g., excipients) in the intermediates, it should be processed and filtered last. Filter S had the highest filtration rate at 43-50 mL/min, followed by Filter E at 41-50 mL/min, and then Filter M at 32-48 mL/min. To properly flush each antigen between filtrations for intermediate formulation, Filter S required 150-200 mL, Filter M required 200 mL and Filter E required >300 mL of buffer. Filter E had the largest capsule holding volume. Filter S was selected as the best choice as it met all criteria, had a higher filtration rate, and used the least amount of flushing volume. Satisfactory results were obtained using the dual Filter M assembly, the dual Filter S assembly, and a dual Filter E assembly. For each filter, the order of proteins was selected randomly with buffer flushing between each protein addition at constant pump speed.

Filter S was then tested for protein loss during filtration of a bivalent formulation; however, no determinable loss of the individual proteins occurred, as Protein A was below targeted concentration by an average of 3.9% and Protein D was above targeted concentration by an average of 9.4% after dual filtration. Target concentration range of final product oft 30% per protein (inclusive of assay variability) was therefore met.

In this small scale system, ingredient addition is based on product specific gravity, desired volumes by weight, and zeroing of bag weight in-line prior to addition. Small bags (IL) in series, such as those containing intermediates, are prone to moving around on scales or balances, leading to inaccuracies when attempting to measure weight in bags. For this system, load cells supported by a post and bracketing assembly were designed to weigh suspended bags during addition. They were selected for their ability to withstand measurement disturbances from side loads (bag swaying) and they have moveable load points, making it convenient to hang bags of different configurations. In addition, according to the manufacturer, the load cells have high individual accuracy with a combined error of 0.02% and repeatability of 0.01%, and designed to discount measurements due to thermal or vibration interference. Bags were primed, tared, and weighed using the device with a microprocessor-based control with display.

Readings from the load cells once ingredients were pumped into the hanging bags had an average percentage difference of ±0.15% (n=35, practical minimum and maximum weights applied) compared with target weight, largely due to human error. Aluminum content samples were taken after intermediate individual adjuvanted proteins and final multivalent formulations with use of hanging load cells. Results were well within acceptable final product limits of 0.28±0.1 mg Al/0.5 mL.

### Example 3

### Study CA-08-010

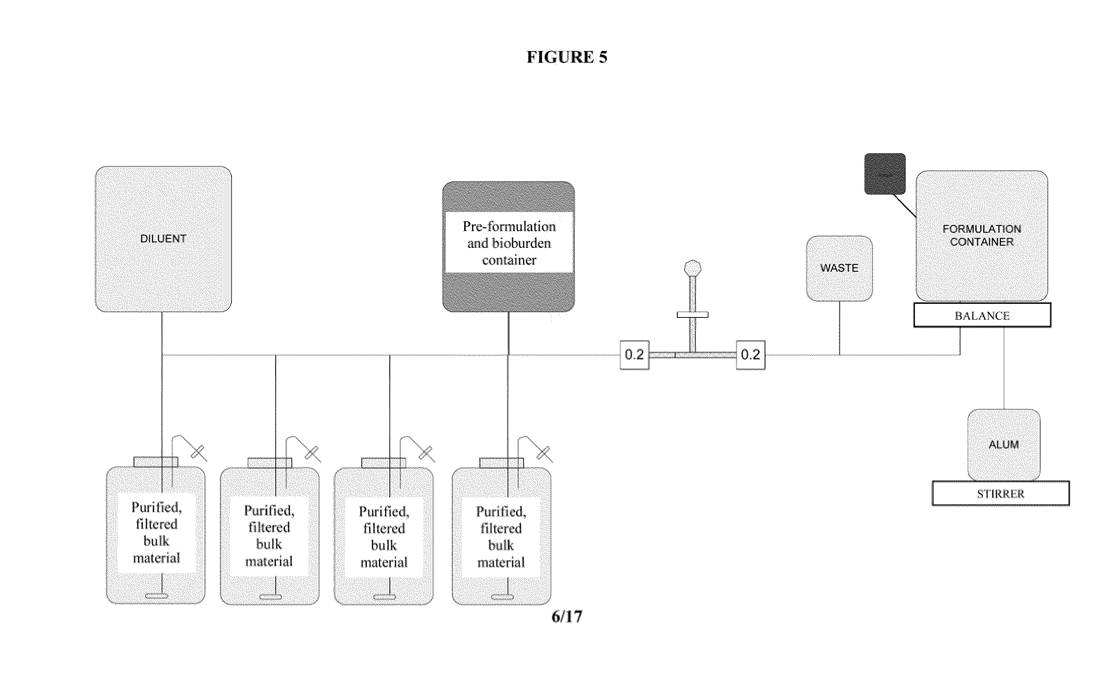

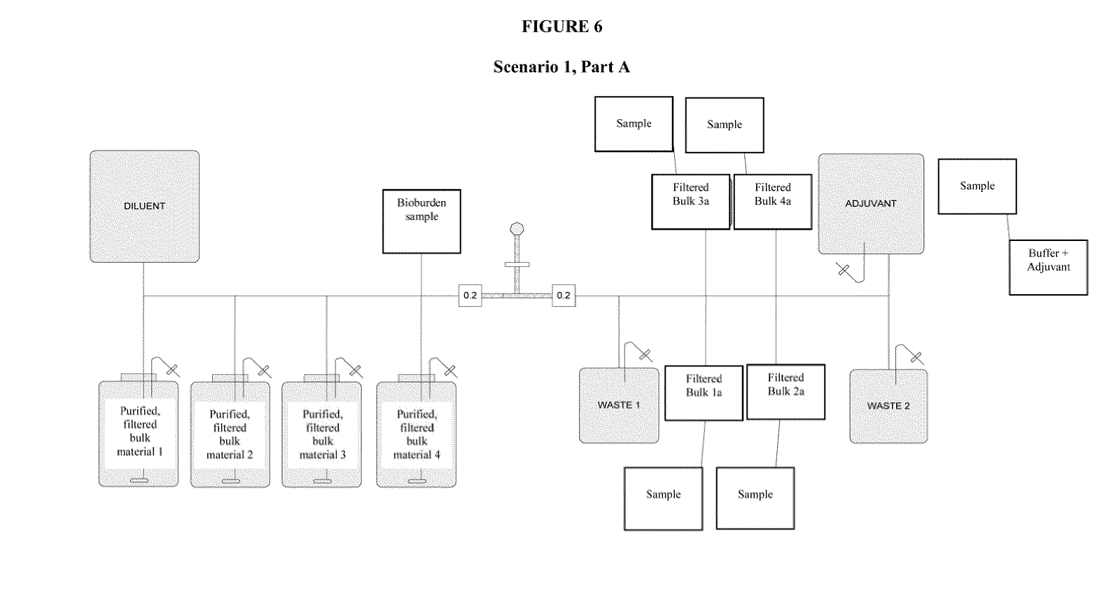

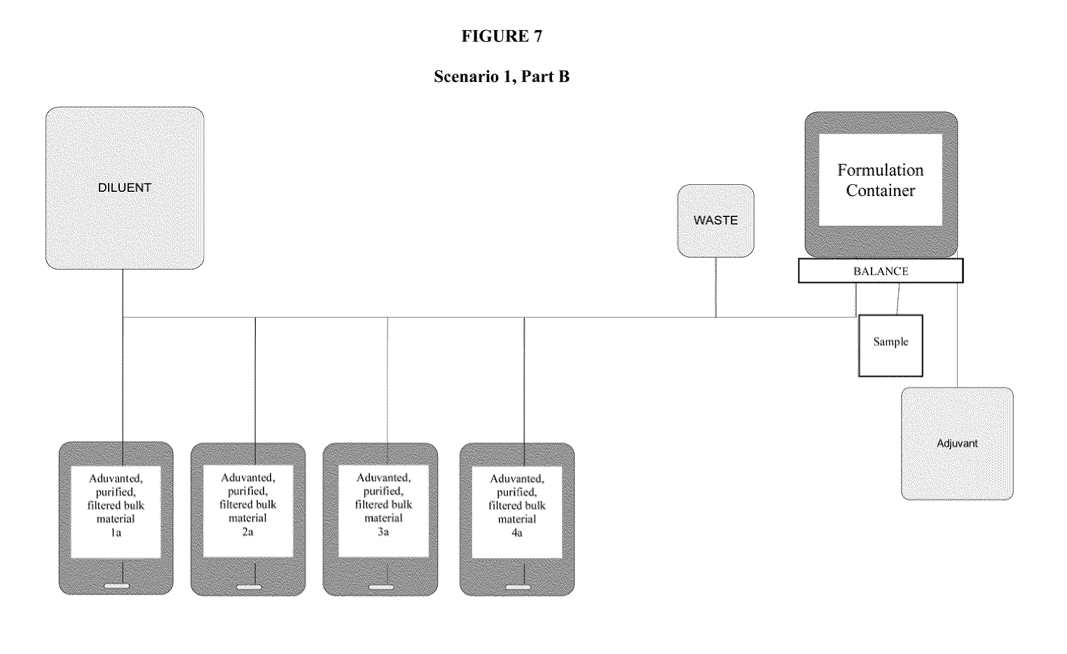

The purpose of this study was to formulate a multivalent product successfully and accurately. The study tested two different scenarios: the “phase 1” process (FIG. 5), which was the same process as the single-valent formulation such that all ingredients are added at one time, versus a “new” process (FIGS. 6 and 7) where intermediates made of stock individual adjuvanted antigen formulations are mixed to allow for binding, then blended in a final step.

Tables 3 and 4 summarize the testing matrix for CA-08-010. ID “A” was used as a control since there was no adjuvant in this formulation. PBS was no longer the buffer of choice, however, an assay had already been developed with this buffer and some of the antigens. For this study final formulation protein concentrations by HPLC, Aluminum content analysis by ICP and particle size by Mastersizer were compared for Scenarios 1 and 2 (IDs “C” and “B”). Aluminum hydroxide (AlOOH) was the adjuvant of choice, however, as the analytical testing lab was still developing the HPLC testing method for AlOOH bound antigens, Scenario 1 was also performed with the previous adjuvant and buffer used, Aluminum phosphate (AlPO₄) and PBS as represented by ID “D”. This way, if the results for ID “C” were skewed or offset, it could be confirmed by ID “D” if it was due to the process or the HPLC assay. Chromatographs were to show any detectable cross-contamination of antigens in the single-valent intermediates.

The antigens used (Proteins A, B, D and C) were prepared at a concentration of 200 μg/mL per intermediate bag for Scenario 1 and final formulated concentrations of 20 μg/mL/protein for all scenarios. The disposable bags used for all scenarios were TC-TECH using C-Flex tubing and Sartopore 2 filters.

TABLE 3
Testing Matrix for CA-08-010

| ID | A | B | C | D |
|---|---|---|---|---|
| Formulation process | Scenario 2 | Scenario 2 | Scenario 1 | Scenario 1 |
| Mixing Parameters | Wave Mixer | Wave Mixer | Wave Mixer | Wave Mixer |
| Adjuvant | Unadjuvanted | AlOOH, target | AlOOH, target | AlPO₄, target |
| | | 1.25 mg/mL | 1.25 mg/mL | 3 mg/mL |
| Buffer | 10 mM Sodium | 10 mM Tris-HCL | 10 mM Tris-HCL | 10 mM Sodium |
| | Phosphate pH 7.2 | Buffer pH 7.4 150 | Buffer pH 7.4 150 | Phosphate pH 7.2 |
| | with 150 mM | mM NaCl (TBS) | mM NaCl (TBS) | with 150 mM |
| | Sodium Chloride | | | Sodium Chloride |
| | (PBS) | | | (PBS) |
| Intermediate Bulk | N/A | N/A | 200 μg/mL (~6x) | 200 μg/mL (~6x) |
| Concentration | | | | |
| (Total protein) | 80 μg/mL | 80 μg/mL | 80 μg/mL | 80 μg/mL |
| Final Formulated | | | | |
| Concentration | | | | |
| Each Antigen | 20 μg/mL · protein | 20 μg/mL · protein | 20 μg/mL · protein | 20 μg/mL · protein |
| Concentration | | | | |
| Sample points | N/A | N/A | 1a, 2a, 3a, 4a after | 1a, 2a, 3a, 4a after |
| | | | 3.5 h mixing | 3.5 h mixing |
| | Final formulation | Final formulation | Final formulation | Final formulation |
| | after 30 min mixing | after 30 min mixing | after 30 min mixing | after 30 min mixing |
| Testing Outcome | Final formulation: | Final formulation: | Intermediates: | Intermediates: |
| | 20 μg/mL · protein | 20 μg/mL · protein | 200 μg/mL · protein | 200 μg/mL · protein |
| | 80 μg/mL total | 80 μg/mL total | Final formulation: | Final formulation: |
| | Suitable fluid path | | 20 μg/mL · protein | 20 μg/mL · protein |
| | (reduced pressure | | 80 μg/mL total | 80 μg/mL total |
| | build-up), experience | | | |

TABLE 4
Observations and Actions from CA-08-010

| Observation | Action |
|---|---|
| Both processes | No action necessary. |
| worked well | |
| Many steps for | Time consuming due to tube welding custom |
| both, time | assemblies onsite. Once process becomes finalized, |
| consuming for | the bag assembly supplier (e.g. Thermofisher) will |
| setup | provide a gamma sterilized pre-made assembly to |
| | minimize setup time. |
| Aggregation | ID “B” was performed before ID “C”, and it was |
| (observed as | believed that possibly the aggregation was due to the |
| white flakes) | order of component addition: proteins, buffer, |
| occurred with | adjuvant. Concerned this may be observed in “C”, |
| Scenario 2, | the order was changed for ID “C” to adjuvant, |
| ID “B” | protein, buffer for the intermediate formulation |
| before mixing | and no aggregation was observed |
| Sedimentation of | Additional mixing studies required to test different |
| adjuvant during | mixing technologies and parameter optimization of |
| mixing using the | Wave Mixer. |
| Wave Mixer | |
| “Flashing” | Flashing occurs when a weld is made unsuccessfully |
| from the tube | between the two pieces of tubing where the contents |
| welder occurred | of the tubing remain integral, however, the fusion |
| | does not leave a sufficient opening for fluid to flow |
| | through the inner welded diameter of the tubing. |
| | Believed to have occurred by using wet tubing or not |
| | “popping” the tubing immediately after a tube weld. |

In order to quantify the dispensing accuracy of the ingredients being added to the formulation, HPLC measured the protein concentration in the intermediates and final formulations. For the intermediate concentrations, AlOOH and AlPO4 adjuvanted protein formulations were within a range of ±30% except for Protein A intermediate which read low for both adjuvanted formulations due to issues with reference standard. New desorption methods are shown.

In these intermediates of the individual stock antigen formulations, it is also important to ensure there is minimal or no residual protein (cross-contamination) from the other antigens during the process that may have been carried into the bags during formulations. Based on the four antigen intermediate formulations tested, there were no measurable residuals, thus confirming purity of single-antigen intermediates. For the AlOOH adjuvanted formulations, it is more difficult to conclude due to desorption issues that resulted in lower concentrations of the antigens as well as shoulders present in the peaks, even for the unadjuvanted formulations (not shown).

The chromatograms of the final formulations prepared as in Scenario 1 displays peaks of the individual antigens present for the formulation with AlPO₄ adjuvantation. For the AlOOH adjuvanted formulations, it is more difficult to conclude due to desorption issues that resulted in lower concentrations of the antigens as well as shoulders present in the peaks, even for the unadjuvanted formulations (not shown).

Using crude testing methods and first-time processing scenarios (1—preadsorbed antigens, 2—antigens adsorbed after blending), AlOOH and AlPO₄ adjuvanted protein final formulations and the “old” desorption method, all samples for both AlOOH and AlPO4 adjuvanted formulations and Scenarios 1 and 2 were within a ±30% range of final formulation per antigen. Using the new desorption method, however, the Protein D samples were recalculated against a new standard curve showing much higher values were obtained. Protein A values are offset due to reference standard discrepancies.

Adjuvant concentration was measured by aluminum content using Inductively Coupled Plasma Atomic Emission Spectrometry as a measure of bulk product homogeneity of suspension. Adjuvant concentrations of intermediate stock and final formulations in bags were within the target ranges (±0.1 mg Al/0.5 mL) for both AlOOH and AlPO₄.

Each intermediate and final formulation tested by Mastersizer showed consistent distribution of particle sizes at 50% distribution and lower for both AlOOH and AlPO4 adjuvanted formulations. The AlPO₄ readings are well within the expected values for the control (5-12 um).

Several studies were performed for mixing optimization of the stock antigen, intermediate and final formulations in disposable bags. Major observations for these studies included observation of overall mixing efficiency, presence of unwanted, visible aggregation, homogeneity, foaming, dead pockets in the bags, and spattering (when there is accumulation of adjuvant on the inner top of the bag). Four different mixing systems were attempted in these studies. Mixing optimization studies were performed by visual observation of 1 L bags containing adjuvanted intermediate and 5 L bags with adjuvant solutions mixed using a Wave Mixer, Recirculation Line, Rotating Drum, and Stir plate.

The Wave Mixer is designed for mixing liquids in disposable bags up to 20 kg using Wave motion technology. For this reason, and because the current system using a stir bar and Stir Plate which are difficult to setup and control, it was tested and optimized more than any of the other systems. A recirculation line was also tested where two tubing lines coming from the bag were welded and looped through a peristaltic pump to keep the line in circulation. A rotating drum (used primarily for rotating syringes) was tested by affixing a 1 L bag to it using cable ties.

### Example 4

### Study CA-07-120

To optimize parameters of the WAVE mixer instrument and to compare mixing effectiveness of the WAVE mixer with the stir plate using AlPO₄ adjuvanted products. The formulation of 3750 ml of 3 mg/ml AlPO₄+20 ug/ml Protein D+PBS in a 5 L TCTECH bag was tested. Wave Mixer operating parameters for 5 L bag at 40 rpm, 6° for 30 min reduces foaming and pooling over other settings as can be seen in Table 5.

TABLE 5
Preliminary Optimal Wave Mixer Settings

| CA-07-120 | Low -6 rpm | Med-25 rpm | High-40 rpm |
|---|---|---|---|
| High-12° | Dead pockets | Dead pockets | Speed excessive |
| | some foaming | some foaming | Excessive foaming |
| | large air | large air | Bag moves around |
| | bubbles | bubbles | |
| Med-6° | Minimal | Incomplete | Minimal adjuvant |
| | mixing | mixing | pooling at bottom |
| | | | layer |
| | | | Air bubbles |
| Low-2° | No mixing | Incomplete | Incomplete |
| | | mixing | mixing |

### Example 5

### Study GA-08-044

To perform visual observations of adjuvanted product mixed using the WAVE mixer instrument. Parameters tested: 10° at 20 rpm for 30 min or 6° at 40 rpm for 30 min.

Formulations: 20 ug/ml Protein B+PBS in ALOOH in 5 L TC-TECH bags at 500 mL and 3000 mL capacity; 20 ug/ml Protein D+PBS in ALOOH in 4×1L TCTECH bags at 200 mL and 750 mL capacity.

Outcomes: Optimal settings with 5 L bag were at 10° at 20 rpm for 30 min or 6° at 40 rpm for 30 min. Settling occurs at both settings for 4×1 L bags. Alum settling occurs more at maximum volumes then at minimum volumes

### Example 6

### Study CA-08-050

Objective: To perform visual observation of concentrated stock AlOOH adjuvant and adjuvanted intermediate bulk formulations mixed using the WAVE mixer, recirculation line, rotating drum, and stir plate. Blue dextran was used to bind to the AlOOH adjuvant for phase separation to identify sedimentation.

Formulations: 1) 5 L TCTECH bag at 200 mL and 750 mL capacities: 24.30 mg/ml AlOOH w/0.01% Blue dextran; 2) 1 L TCTECH bag at 200 mL and 750 mL capacities: 1.25 mg/ml AlOOH w/200 ug/ml Protein A w/0.005% Blue dextran in TBS.

Mixing time: up to 30 minutes

Assays: 1. Visual inspection 2. Aluminum content analysis

Outcome: Wave Mixer; operating parameters were at 40 rpm, 6° for both 5 L and 1 L bags.

TABLE 6
Wave Mixer Mixing Efficiencies in 1 L and 5 L bags

| | | | Homogeneous | | | |
|---|---|---|---|---|---|---|
| | Good Mixing | No | (uniphase, no | Minimal | No Dead | |
| | (low shear) | Aggregation | settling) | Foaming | Pockets | Spattering |
| 5 L Bag | ✓ | ✓ | ✓ | ✓ | ✓ | |
| high volume | | | | | | |
| 5 L Bag | ✓ | ✓ | ✓ | ✓ | ✓ | |
| low volume | | | | | | |
| with clamp | | | | | | |
| 1 L Bag | ✓ | ✓ | ✓ | | ✓ | |
| high volume | | | | | | |
| 1 L Bag | ✓ | ✓ | ✓ | ✓ | ✓ | ✓ |
| low volume | | | | | | |

Recommendations resulting from these studies: 1) Tap 5 L bag occasionally to break spattering; 2) Use clamp when 5 L bag is at low volumes to reduce dead pockets.
Recirculation Line: operating parameters at fastest pump speed (10)

TABLE 7
Recirculation Line Mixing Efficiencies in 5 L bags

| | | | Homogeneous | | | |
|---|---|---|---|---|---|---|
| | Good Mixing | No | (uniphase, no | Minimal | No Dead | |
| | (low shear) | Aggregation | settling) | Foaming | Pockets | Spattering |
| 5 L Bag | ✓ | ✓ | ✓ | ✓ | | slight |
| high volume | | | | | | |
| 5 L Bag | ✓ | ✓ | ✓ | ✓ | | ✓ |
| low volume | | | | | | |
| with clamp | | | | | | |

Recommendations from these studies: 1) 5 L bags must be suspended slightly angled from the vertical hanging position to avoid tubing from folding, and affecting flow rate; 2) pump should be tested at slower speeds for optimization; 3) 1 L bags not tested as each intermediate bag would require a recirculation line and this would take up a significant amount of processing area, time and setup. It would also be difficult to control; 4) use clamp when 5 L bag to reduce dead pockets.

### Rotating Drum

Refer to Table 8; operating parameters at highest RPM on unit.

TABLE 8
Rotating Drum Mixing Efficiencies in 1 L bags

| | | | Homogeneous | | | |
|---|---|---|---|---|---|---|
| | Good Mixing | No | (uniphase, no | Minimal | No Dead | |
| | (low shear) | Aggregation | settling) | Foaming | Pockets | Spattering |
| 1 L Bag | ✓ | ✓ | ✓ | | ✓ | ✓ |
| high volume | | | | | | |
| 1 L Bag | ✓ | ✓ | ✓ | ✓ | ✓ | ✓ |
| low volume | | | | | | |

Recommendations from these studies: 1) time consuming and difficult to setup due to cable tying and wheel configuration; 2) drum should be tested at slower speeds for optimization; and, 3) significant foaming when bags at high volume.

### Stir Plate

Refer to Table 9; operating parameters at 400 RPM.

TABLE 9
Stir Plate Mixing Efficiencies in 1 L bags and 2 L and 5 L Bottles

| | | | Homogeneous | | | |
|---|---|---|---|---|---|---|
| | Good Mixing | No | (uniphase, no | Minimal | No Dead | |
| | (low shear) | Aggregation | settling) | Foaming | Pockets | Spattering |
| 1 L Bag | ✓ | ✓ | ✓ | ✓ | ✓ | ✓ |
| high volume | | | | | | |
| 1 L Bag | ✓ | ✓ | ✓ | | ✓ | ✓ |
| low volume | | | | | | |
| 5 L Bottle | ✓ | ✓ | ✓ | | ✓ | ✓ |
| (control) | | | | | | |
| 2 L Bottle | ✓ | ✓ | ✓ | | ✓ | ✓ |
| (intermediate | | | | | | |
| formulation | | | | | | |
| control) | | | | | | |

Recommendations from these studies: As foaming was observed at low volumes, test at lower speeds and low volumes to reduce foaming.

The aluminum content results in all mixing systems tested for both 5 L and 1 L bags were within 90% of the control (stir plate using glass bottle with stir bar). This is well within the aluminum content release criteria for final product which is ±0.1 mg Al/0.5 mL. Therefore aluminum content results were consistent with all mixing systems showing good homogeneity in bags.

From these results, it was determined that the rotating drum created too much foaming (even without a surfactant) and the recirculation line was difficult to setup to ensure all parts of the bag were recirculating efficiently. The stir plate and Wave Mixer were successful though the speed on the stir plate required optimization as 400 rpm created too much foaming at lower volumes.

### Example 7

### Study CA-08-065

To further optimize parameters of the WAVE mixer instrument by mixing adjuvanted intermediate in 1 L bag with a worst case formulation. According to CA-07-120 and CA-08-044 study, most optimization settings of the WAVE mixer instrument were performed for low concentrated formulations and without Tween. For mixing of an adjuvant intermediate in 1 L bag, a worst case formulation was defined for an adjuvanted intermediate with Tween 80, higher protein concentration and aluminum hydroxide. This study will determine optimal WAVE mixer parameters for said worst-case adjuvanted intermediate in 1 L bag at high and low volumes.

Formulation: Adjuvanted intermediate (1.25 mg/mL AlOOH, 400 μg/ml protein, 0.05% Tween 80 in TBS) in 1 L bag. Order added: Adjuvant, protein, Tween 80, and TBS.

Table 10 describes the results from the angles and speed of the Wave Mixer tested.

TABLE 10
Wave Mixer Pitch and Speed For 1 L Bags with ALOOH,
0.05% Tween 80, and ProteinA Protein in TBS
Speed

| | Low -10 | Med - 20 | Med - 30 | High - 40 |
|---|---|---|---|---|
| Pitch | rpm | rpm | rpm | rpm |
| High-12° | Not | Not | Some foaming | High |
| | efficient | efficient | No adjuvant | foaming |
| | mixing | mixing | settling occurred. | |
| | | | No dead pockets. | |
| | | | Mix completely. | |
| Med-10° | | | Not efficient | High |
| | | | mixing | foaming |
| Low-6° | | | | Not |
| | | | | efficient |
| | | | | mixing |

Optimal mixing settings for a 1 L bag with worst-case intermediate formulation was at 30 rpm with a 12° tilt.

### Example 8

### Study CA-08-064

This study was performed to evaluate the effect of mixing processes (stir bar and wave mixer) of adjuvanted and non-adjuvanted products, perform visual observation, and characterize aggregation & foaming of Tween 80 using these mixing processes. Protein C antigen required addition of a surfactant such as Tween 80 in order to reduce the potential for aggregation, however, Tween 80 has a tendency to increasing foaming during mixing (stirring and/or shaking). This study represents the worst case situation with an antigen prone to aggregation and the presence of the foaming surfactant added to the intermediate and final formulations to evaluate whether the optimized mixing process from previous studies is also applicable.

After reviewing information provided by Wave Biotech (Source: Singh, 2000), it was confirmed that sedimentation had occurred in CA-08-010 in the 1 L bags due to pooling at the bottom center of bag where the tilt angle for these smaller bags was not enough to create a sufficient velocity for movement of the adjuvant particulates to travel any great distance (e.g. caught in momentum of the wave). For this reason, different parameters on the Wave Mixer were tested for the 1 L bags as the bag configuration and geometry is different for the longer, larger 5 L bags though the same settings had been used in the past.
Formulation: Alhydrogel: 24.35±2.43 mg/ml ALOOH.
Tris Buffered Saline (TBS):
protein antigen: 1) Protein A 796.6 ug/ml; 2) Protein DO 244.2 ug/ml); 3) Protein C, 381.72 ug/ml.
2% TWEEN80 in TBS: Lot#10002884-199-EX
Assays: 1. Visual inspection; 2. Aluminum content analysis
Outcomes: Table 11 describes the mixing efficiencies with Wave Maxer and Stir Plate for 1 L and 5 L bags at the various settings tested.

TABLE 11
Mixing Efficiency of 1 L and 5 L bags with Wave Mixer and Stir Plate at Various Settings

| | | | | | |
|---|---|---|---|---|---|
| | | Homogeneous | | | |
| Good Mixing | No | (uniphase, no | Minimal | No Dead | |
| (low shear) | Aggregation | settling) | Foaming | Pockets | Spattering |

Intermediate formulation unadjuvanted: 200 ug/ml Protein C w/0.05% Tween 80 in TBS

| | | | | | | |
|---|---|---|---|---|---|---|
| Wave Mixer- | ✓ | ✓ | ✓ | ✓ | ✓ | ✓ |
| 30 rpm/12°, | | | | | | |
| 750 ml in 1 L bag | | | | | | |
| Wave Mixer- | ✓ | ✓ | ✓ | ✓ | ✓ | ✓ |
| 30 rpm/12°, | | | | | | |
| 200 ml in 1 L bag | | | | | | |
| Stir Bar-200 rpm | Not | | | | | |
| 750 ml in 1 L bag | efficient | | | | | |
| | mixing | | | | | |
| Stir Bar-300 rpm | ✓ | ✓ | ✓ | ✓ | ✓ | ✓ |
| 750 ml in 1 L bag | | | | | | |
| Stir Bar-400 rpm | ✓ | ✓ | ✓ | ✓ | ✓ | ✓ |
| 750 ml in 1 L bag | | | | | | |
| Stir Bar-200 rpm | Not | | | | | |
| 200 ml in 1 L bag | efficient | | | | | |
| | mixing | | | | | |
| Stir Bar-300 rpm | ✓ | ✓ | ✓ | | | ✓ |
| 200 ml in 1 L bag | | | | | | |
| Stir Bar-400 rpm | ✓ | ✓ | ✓ | | ✓ | ✓ |
| 200 ml in 1 L bag | | | | | | |

Intermediate formulation adjuvanted: 200 ug/ml Protein
C w/0.05% Tween 80 in TBS, 1.25 mg/ml ALOOH)

| | | | | | | |
|---|---|---|---|---|---|---|
| Wave Mixer- | ✓ | ✓ | ✓ | | ✓ | ✓ |
| 30 rpm/12°, | | | | | | |
| 750 ml in 1 L bag | | | | | | |
| Wave Mixer- | ✓ | ✓ | ✓ | | ✓ | ✓ |
| 30 rpm/12°, | | | | | | |
| 200 ml in 1 L bag | | | | | | |
| Stir Bar-200 rpm | Not | | | | | |
| 750 ml in 1 L bag | efficient | | | | | |
| | mixing | | | | | |
| Stir Bar-300 rpm | ✓ | ✓ | ✓ | Less | ✓ | ✓ |
| 750 ml in 1 L bag | | | | foam | | |
| Stir Bar-400 rpm | ✓ | ✓ | ✓ | More | ✓ | ✓ |
| 750 ml in 1 L bag | | | | foam | | |
| Stir Bar-200 rpm | Not | | | | | |
| 200 ml in 1 L bag | efficient | | | | | |
| | mixing | | | | | |
| Stir Bar-300 rpm | ✓ | ✓ | ✓ | ✓ | Use bag | ✓ |
| 200 ml in 1 L bag | | | | | clamp | |
| Stir Bar-400 rpm | ✓ | ✓ | ✓ | | ✓ | ✓ |
| 200 ml in 1 L bag | | | | | | |

Final formulation adjuvanted: 1.25 mg/ml ALOOH, 100 ug/ml Protein
A & Protein D, 50 ug/ml Protein C, w/0.05% T80 in TBS)

| | | | | | | |
|---|---|---|---|---|---|---|
| Wave Mixer | ✓ | ✓ | ✓ | | ✓ | ✓ |
| 40 rpm/6° | | | | | | |
| 3750 ml in 5 L bag | | | | | | |
| Stir Bar-400 rpm | | ✓ | | ✓ | ✓ | ✓ |
| 3750 ml in 5 L bag | | | | | | |
| Wave Mixer- | ✓ | ✓ | ✓ | ✓ | ✓ | ✓ |
| 20 rpm/10° | | | | | | |
| 3750 ml in 5 L bag | | | | | | |
| Wave Mixer- | ✓ | ✓ | ✓ | | ✓ | ✓ |
| 30 rpm/10° | | | | | | |
| 3750 ml in 5 L bag | | | | | | |
| Stir Bar-500 rpm | ✓ | ✓ | ✓ | | ✓ | ✓ |
| 3750 ml in 5 L bag | | | | | | |

Aluminum Content Analysis: The aluminum content results in all mixing systems tested for both intermediate 1 L bags and final formulations in 5 L bags were within 90% of the control (stir plate using glass bottle with stir bar). This is well within the aluminum content release criteria for final product which is ±0.1 mg Al/0.5 mL. Therefore aluminum content results were consistent with all mixing systems showing good homogeneity in bags.

The overall summary for mixing studies is shown below for the formulations tested. The best parameters can be seen in the tables above in this section of the report. Mixing efficiency, homogeneity, foaming, dead pockets and spattering is dependent on: 1) the mixing system selected; 2) mixing system parameters (e.g. rpm, tilt angle); 3) bag size and shape; 4) formulation ingredients and concentrations; and, 5) configuration of the bag as it is placed on/in the mixing system (e.g. vertical, bag clamp). For future formulations (those tested) with the 5 L bag, the Wave Mixer will be used as the preferred mixing system as this provided the best results. For intermediate formulations with the 1 L bag, either the Wave mixer or the stir plate should be used as the rotating drum is difficult to setup and caused foaming without testing with Tween 80 in the formulation. However, should mixing while blending of the intermediates be required, only the stir plates should be used due to processing setup limitations. Table 12 summarizes the mixing efficiency results from studies CA-07-120, CA-08-044, CA-08-050, CA-08-064, CA-08-065 for various systems tested with single-use disposable bags. The Wave Mixer produced best results for both 1 L intermediate and 5 L final formulation bags.

Tnble 12

| | | | | |
|---|---|---|---|---|
| | Homogenous | | | No |
| No Visible | (uniphase, | Minimal | No Dead | Spat- |
| Aggregation | no setting) | Foaming | Pockets | tering |

Containers on Stir Plates

| | | | | |
|---|---|---|---|---|
| 1 L | ✓ | ✓ | ✓ | ✓ |
| intermediate | | | | |
| bag | | | | |
| 5 L bag | ✓ | 500 rpm OK | ✓ | ✓ |
| 5 L bottle | ✓ | ✓ | ✓ | ✓ |
| (control) | | | | |
| 2 L | ✓ | ✓ | ✓ | ✓ |
| intermediate | | | | |
| bottle | | | | |
| (control) | | | | |

Recirculating Line

| | | | |
|---|---|---|---|
| 5 L bag | ✓ | ✓ | ✓ |

Rotating Drum

| | | | | |
|---|---|---|---|---|
| 1 L | ✓ | ✓ | ✓ | ✓ |
| intermediate | | | | |
| bag | | | | |

Wave Mixer

| | | | | | |
|---|---|---|---|---|---|
| 5 L bag | ✓ | ✓ | ✓ | ✓ | ✓ |
| 1 L | ✓ | ✓ | ✓ | ✓ | ✓ |
| intermediate | | | | | |
| bag | | | | | |

### Example 9

### Filtration Studies

Filtration studies have been performed with the multivalent antigens. Three filters have been tested and compared to determine which one is the most suitable for the filtration of the five test proteins: Millipak 20 from Millipore, Sartopore 2 from Sartorius and EBV from Pall. Up to five antigens were filtered through the same filter and buffer was flushed through the filter between each protein filtration to remove the residual proteins from the filter for intermediate bulk stock antigens. The different parameters that are analyzed during the process were the pressure in the filter, the flow rate and the amount of protein in the wash buffer. The data obtained enabled us to determine the best order of filtration, the volume of buffer necessary to remove the proteins from the filter, and the decay ratio of the proteins. Further analysis enabled to determine the loss of protein in the filters during the filtration.

The objective of this study was to compare three filters regarding the filtration of the five test antigens, and to determine which filter(s) is (are) most suitable for the filtration of the test antigens, based on the pressure in the filter during the filtration, the decay ratio, the protein loss and the volume of buffer necessary to flush the filters. An increase of pressure in the filter could indicate a plugging of the pores and thus aggregation of the proteins at the filter face or interaction between the protein and the filter (Sharma et al., 2008).

The study also determined the optimal order of filtration for each filter and confirm if there was a detectable amount of protein lost in the filter. The scope of this study included the filtration of five antigens: Protein A, Protein D, Protein E, Protein Band Protein C. TBS-Tween 80 (2%). Small scale studies were first performed to determine the order of filtration for the antigens. The antigens were then sterile-filtered at full scale through three different filters: Millipak 20 (Millipore, part #MPGL02GH2), EBV (Pall, part #1EBV7PH4) and Sartopore 2 (Sartorius, part #5441307H4G). Millipak 20, EBV and Sartopore 2 have been recommended by the suppliers for multi-antigen filtration, based on two critical specifications: 1) the surface filtrations were adapted for the volume of protein solutions and buffer to be filtered; and, 2) the PVDF or PES membranes and the geometries are suitable for the filtration of recombinant proteins which can be up to 100 000 Daltons in size. Direct comparison filtration studies and bacterial retention studies were performed. Millipak 20 disposable filter units are stacked disc filters designed for the removal of particles and microorganisms from liquids and gases.

TABLE 13
Millipak 20 Specifications

| | |
|---|---|
| Support Material | Polycarbonate |
| Configuration | Stack Disk |
| Vent Cap Material | PVDF |
| Filter Brand Name | Durapore |
| Bubble Point at 23° C. | ≧3450 mbar (50 psig) air with water |
| Max Inlet Pressure | 5.2 bar at 25° C. |
| Process Volume | 10 L |
| Connections, Inlet/Outlet | 6 mm (¼ in.) Hose Barb with bell |
| Filter pore size | 0.22 μm |
| Max Differential Pressure | 4.1 bar @ 25° C.; 1.7 bar @ 80° C.; 0.35 @ |
| | 123° C. |
| Capsule Type | Liquid |
| Filtration Area | 100 cm² |
| Filter Material | Hydrophilic PVDF |
| Flow rate | 1.5 L/min @ 1.75 bar P |

Sartopore 2-γ-capsules (Sartorius Stedim Biotech; Table 14) are 0.2 μm rated sterilizing grade filter capsules designed for connection to flexible-bag-container-systems prior to sterilization by γ-irradiation.

TABLE 14
Sartopore 2 Specifications

| | |
|---|---|
| Support Material | Polypropylene |
| Vent Cap Material | Polyethersulfone |
| Filter pore size | 0.45 + 0.22 μm |
| Max Differential Pressure | 4 bar at 20° C.; 2 bar at 80° C. |
| Capsule Type | Liquid |
| Filtration Area | 150 cm² |
| Filter Material | Polyethersulfone, assymetric |

Pall's Mini Kleenpak sterilizing capsule filters are compact pharmaceutical-grade capsule filters featuring low hold-up volumes.

TABLE 15
EBV Specifications

| | |
|---|---|
| Support Material | Polypropylene |
| Max Operating Pressure | 4.1 bar at 38° C.; 2.1 bar at 80° C. |
| Filter pore size | 0.45 μm + 0.22 μm |
| Capsule Type | Liquid |
| Filtration Area | 200 cm² + 220 cm² |
| Filter Material | PVDF + PES |
| Flow rate | 322 mL/min/100 mbar |

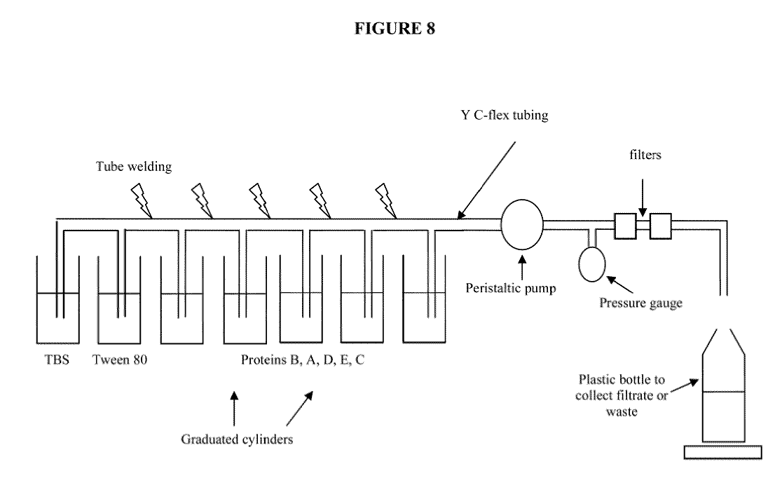

A double filtration was performed to satisfy GQD recommendations of having a “redundant” sterilizing grade filter (GQ_{—}000795). All the antigens were filtered through the same filter and collected separately after filtration. Between each protein filtration, the filters were flushed with buffer in order to remove the protein from the filters. A pressure gauge and the measurement of the flow rate were used to determine if there is any increase of pressure during the filtration, e.g. if the filter starts to clog. The assembly is illustrated in FIG. 8. The wash fractions, after each protein filtration, were analyzed by BCA to determine the volume of buffer necessary to remove the proteins from the filters.

The reagents used for this study are:
3×5 L of TBS buffer (Tris 10 mM with NaCl 150 mM), pH 7.4. lot #C12431
700 mL of Protein A protein solution, 979 μg/mL,
500 mL of Protein D protein solution, 1311 μg/mL,
200 mL of Protein D protein solution, 1143 μg/mL,
700 mL of Protein E protein solution, 848 μg/mL,
700 mL of Protein B protein solution, 1038 μg/mL,
700 mL of Protein C protein solution, 491 μg/mL,
Tween 80, batch #C001766,

The materials used for this study included:
15× sterile plastic bottles (250 mL) for the filtered proteins
60× sterile plastic bottles (60 mL) for the flushing buffer
6×250 mL sterile graduated cylinders
1×500 mL container to collect waste
1× Pressure gauge
3× sterile C-flex tubing to be connected to the pressure gauge
6× C-flex Y tubing
Balance scale
Stopwatch
7× tubing clamps
Several sterile powder-free surgical gloves, polyester or disposable Tyvek lab coat, safety glasses
2× Millipak 20 filter, part #MPGL02GH2
2× Sartopore 2 filter, part #5441307H4G
2× EBV filter, part #KA02EBVP2S

BCA assays were performed on the wash buffer samples, as per instructions. The protein solutions, the buffer fractions and the TBS-Tween 80 were collected after filtration to be analyzed by BCA. A sample of the proteins solutions and TBS-Tween 80 solution before filtration was collected. The samples were stored at 2-8° C.

The equipments used for this study included:
Biocontainment hood: equipment BCC1066, functional location B93BCC00009, tech ID B67BCC005, environmental H.V.A.C. performance certification date 22 Feb. 2008
Peristaltic pump: Easyload Masterflex L/S standard drive, model 7518-00
Tube welder: ID #TUW1010, bld93 room 121, certified on 4 Feb. 2008

Testing with the Milliex (Millipak) disc filter showed that filtration throughout capacity of the antigen component was susceptible to its position in the filtration sequence. The five antigens and the TBS-Tween 2% were filtered through the same filters.

The volume filtered through the Millipak 20 filter as a function of time for each antigen was acceptable. The pressure applied by the peristaltic pump is constant during the filtration of the five antigens. The flow rate is constant during the filtration for all the proteins. There is no increase of pressure. The amount of protein in the wash fractions, for each antigen, was acceptable.

The volume of buffer necessary to remove the protein from the filters down to a level lower than the limit of detection is ≧200 mL for Protein A, Protein D, Protein E and Protein B. The less concentrated sample in the calibrating curve is 20 μg/mL, so the amount of protein in a sample is considered insignificant when the concentration is below 20 μg/mL. Due to the presence of Tween 80 in Protein C wash fractions, and because of the interference of the Tween 80 in the BCA assay, HPLC analyses was performed on Protein C wash fractions.

The volume of protein solutions filtered through the Sartopore 2 filter as a function of time was acceptable. The flow rate is constant during the filtration of all the proteins. There is no increase of pressure. The amount of protein in the wash fractions, for each antigen, was acceptable. The volume of buffer necessary to remove the protein from the filters down to a level lower than the limit of detection is ≧150 mL for ProteinA, Protein D and ProteinE, and ≧200 mL for ProteinB. Due to the presence of Tween 80 in Protein C wash fractions, and because of the interference of the Tween 80 in the BCA assay, HPLC analyses was performed on Protein C wash fractions. The volume of protein solutions filtered as a function of time was acceptable. The flow rate is constant during the filtration of all the antigens. The pressure does not increase. The amount of protein in the wash fractions, for each antigen, was acceptable. The volume of buffer necessary to remove protein from the filters down to a level lower than the limit of detection is ≧300 mL for ProteinA, ProteinD, Protein E and ProteinB.

The following experiment was performed to determine the protein loss in the Sartopore 2 filter during the filtration. Protein Aand Protein D were filtered through two Sartopore 2 filters and diluted with TBS to a target concentration of 100 μg/mL. Three runs have been performed for each protein dilution. The diluted samples were then analyzed by BCA to determine the concentration (Table 16).

TABLE 16
Sartopore 2 - Protein Assay Loss

| | Concentration of Protein | Concentration of Protein |
|---|---|---|
| Run # | A (μg/mL) | D (μg/mL) |
| Starting material | 816.76 ± 16.32 | 894.64 ± 48.07 |
| 1 | 99.64 ± 3.78 | 102.33 ± 7.22 |
| 2 | 92.56 ± 2.68 | 120.56 ± 2.02 |
| 3 | 96.32 ± 3.37 | 105.28 ± 4.56 |
| Average | 96.14 ± 4.23 | 109.39 ± 9.5 |

For Protein A, the concentration obtained for the three runs were below the targeted concentration by an average of 3.9%. For Protein D, the concentration obtained for the three runs were above the targeted concentration by an average of 9.4%. Considering the three runs for each protein all together, the targeted concentration of 100 μg/mL is within the interval given by the standard deviation. Thus there is no significant loss of protein in the filter during the filtration of Protein A and Protein D through the Sartopore 2 filter.

As observed in these experiments:
The three filters did not clog during the antigens filtration, so all three filters can be used for the filtration of the antigens.
There is no increase of pressure in the filters during the filtration of the five antigens, for the 3 filters tested. Thus the order of filtration is not relevant. The only limitation is that Protein C should be filtered last, if Tween 80 will be used in the process. Some proteins such that have been reported as being stickier than other proteins, should be filtered last.
Sartopore 2 requires 150-200 mL of buffer, Millipak 20 requires 200 mL and EBV requires 300 mL. Thus Sartopore 2 would be the best choice regarding the wash volumes.
There is no loss of protein during the filtration of Protein A and Protein ID through the Sartopore 2 filters.

Based on these results, the optimal primary filter of choice to use in the multivalent formulation is the 2× Sartopore 2 filters in series because it limits the loss of protein during the filtration and the volume of flushing buffer. An exemplary, suitable alternative is the Millipak 20 filter due to a limited volume of flushing buffer required, however studies would be required to confirm loss of any protein during filtration.

### Example 10

### Study CA-08-077

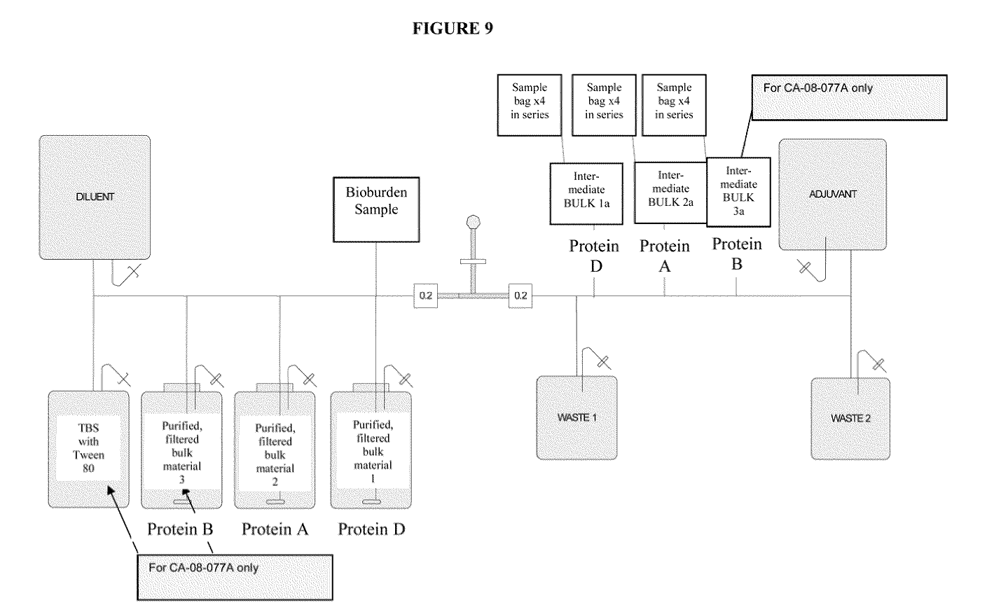

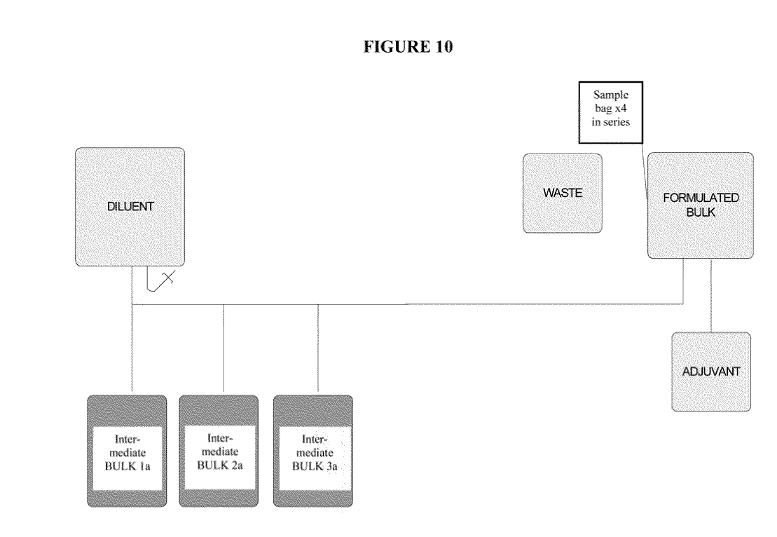

The following study uses parameters optimized in the sections to follow: 1) mixing parameters: Wave Mixer for 5 L final formulations and stir plate for 1 L intermediate bags; 2) filtration: 2×0.2 urn Sartopore 2 filters in series with 150 mL buffer flushing volumes; 3) Process Scenario 1 (FIGS. 9 and 10): preadsorbed intermediates and final blending. Formulation ingredients include: Protein D, Protein A, Protein C and AlOOH Adjuvant (for adjuvanted formulations), TBS, and Tween 80 (for Protein C antigen). The objective of this study was to formulate a multivalent product successfully and accurately and to optimize mixing time of multivalent products, and to ensure homogeneous product throughout Beginning, Middle, and End sampling of final container, and a suitable seal using flip-off caps. Bivalent and trivalent formulations were made with adjuvant. The trivalent was also formulated in unadjuvanted form. Formulation ingredients included: 1) Protein D Protein—Purified concentration values based on HPLC assay; 2) Protein A Protein—Purified concentration values based on HPLC assay; 3) Protein C Protein—Purified concentration values based on HPLC assay; 4) 5 L TC-TECH bag with pooled AlOOH at 23.34 mg/mL (concentration may vary slightly (20.01-24.45 mg/mL) depending on CofA); 5) 5 L 10 mM Tris-HCL Buffer pH 7.4 150 mM NaCl; and, 6) Tween 80, Plant origin, EP grade. Table 17 describes the study formulation matrix.

TABLE 17
CA-08-077 Sampling Matrix

| ID | CA-08-077-A | CA-08-077-B | CA-08-077-C |
|---|---|---|---|
| Formulation | Scenario 1 | Scenario 1 | Scenario 2 |
| process | Trivalent (adj) | Bivalent (adj) | Trivalent (unadj) |
| Mixing | Wave Mixer settings for | Wave Mixer settings for | Wave Mixer settings for |
| Parameters | 5 L final formulation: 20 | 5 L final formulation: 20 | 5 L: 20 rpm, 10° |
| | rpm, 10° | rpm, 10° | |
| | Stir Plate settings for 1 L | Stir Plate settings for 1 L | |
| | during final formulation: | during final formulation: | |
| | 400 rpm | 400 rpm | |
| Adjuvant | AlOOH, target 1.25 | AlOOH, target 1.25 | N/A |
| | mg/mL | mg/mL | |
| Buffer | TBS, pH 7.4, with 0.05% | TBS, pH 7.4 | TBS, pH 7.4, with 0.05% |
| | Tween 80 | | Tween 80 |
| Intermediate Bulk | 400 μg/mL | | N/A |
| Concentration | | | |
| Each Antigen | 20 μg/mL · protein | 20 μg/mL · protein | 20 μg/mL · protein |
| Concentration | | | |
| Sample points | Intermediate bags 1a, 2a, 3a after | | N/A |
| | 30 min, 1 h and 3.5 h mixing | | |
| | Final formulation after 30 | Final formulation after | Final formulation after |
| | min, 1 h and 3.5 h mixing | 30 min mixing | 30 min mixing |
| Testing Outcome | Intermediates: | Final formulation: | Final formulation: |
| | 400 μg/mL · protein | 20 μg/mL · protein | 20 μg/mL protein |
| | Final formulation: | 40 μg/mL total | 60 μg/mL total |
| | 20 μg/mL · protein | | |
| | 60 μg/mL total | | |

Assays: Samples were be tested for total protein concentration by: 1) RP-HPLC—total protein assay+individual proteins; SDS-PAGE and % Adsorption; 2) HPLC—total protein assay+individual proteins (PD CA); 3) Aluminum Content (Bodycote); and, 4) Stability Testing. The desired target accuracy was ±15% (inner target), and the desired Release Testing accuracy (outer target) was ˜±30%. The Intermediate Formulation was to be >400 μg/mL, and the Final Formulation >20 μg/mL/protein. The target aluminum content in ALOOH was 0.28±0.10 mg Al/0.5 mL.

Formulation steps of the intermediates and final formulation for the adjuvanted trivalent are shown below as this process is most complex as compared with unadjuvanted. The bivalent adjuvanted final formulation was made using the intermediates of Protein A and Protein D from the trivalent and the only difference is Part B of the procedure where only two intermediates were used.

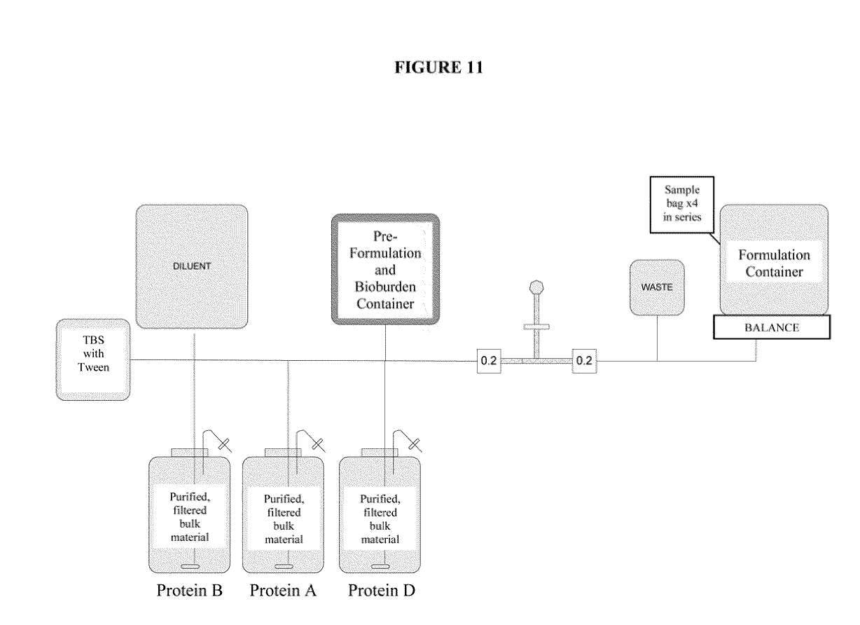

CA-08-077-A—Scenario 1, Trivalent (adj)
1) Set up formulation bag manifold system and formulate to 3750 mL (75% of 5 L bag) and according to BPR 300-FF-04 where possible (FIG. 11, representing sampling locations). Perform only if pre-filter integrity testing is required.
2) Calibrate load cells.
3) Scenario 1, Part A (Note: filtration is performed using 2 in-line 0.2 um Sartopore 2 filters):
   a. Make all appropriate weld connections.
   b. Prime proteins just downstream of each respective “T” junction of manifold. Ensure minimal air bubbles in line.
   c. Prime Tween 80 in TBS to respective “T” junction of manifold. Ensure minimal air bubbles in line.
   d. Prime main line with diluent to priming start mark of bioburden bag and pull ˜10 mL bioburden from diluent to bioburden bag.
   e. Flush main line to Waste 2 with approximately 200 g diluent.
   f. Pre-FIT (not performed for study). Allow diluent to be pushed into Waste bag 1 during FIT.
   g. Prime diluent to each intermediate bag “fill start” mark.
   h. Flush main line to Waste 1 with 200 g adjuvant.
   i. Add required amount of adjuvant to each individual intermediate bag.
   j. Flush main line with 200 g buffer to waste bag 2.
   k. Start with concentrated, purified protein container (Container 1) closest to filter assembly and add required amount of protein to respective intermediate bag starting with Bag 1a (closest to filter assembly).
   l. Pull 10 mL of protein into bioburden sampling bag.
   m. Add required amount of buffer to intermediate bag (must be >150 mL or additional flushing will be required up to 150 mL).
   n. Repeat steps k to m for second protein Container 2/Bag 2a and so on. For Protein C protein requiring Tween 80 perform the following steps:
      i. Repeat step k and l for ProteinC
      ii. Add required amount of Tween 80.
      iii. Pull 10 mL Tween 80 into bioburden sampling bag.
      iv. Add required amount of diluent.
   o. Seal adjuvant container and line just between waste bag 1 and bag 1a.
   p. Perform Post FIT.
4) Sampling
   a. Stir using Wave Mixer for 30 min, 1 hour and 3.5 hours, sample up to 50 mL using sample bags at each time point for each intermediate. Sampling must be clone when intermediates are in suspension. Ensure sampling line is flushed into a waste container (last 50 mL bag) prior to each in-series sample time point.
   b. Sampling will be done in gamma irradiated sampling bags. From these bags, they will be loaded aseptically into standard 3 mL serum vials at sampling 3× each under a GLP hood (except for container integrity) at a volume of approximately 2.5 mL/vial.
   c. At minimum, aluminum content and protein concentration will be tested for each sample point.
5) Storage: After mixing, store intermediates at 2-8 C until required for Part B. Record in/out storage time/date.
6) Scenario 1, Part B
   a. Make all appropriate weld connections except adjuvant can be welded at time of adjuvant addition.
   b. Mix intermediate bags for 30 min each on stir plates at 400 rpm prior to drawing from each of them.
   c. Prime main line with 50 mL of 1a intermediate bag to waste.
   d. Prime main line with 50 mL of 2a intermediate bag to waste.
   e. Prime main line with 50 mL of 3a intermediate bag to waste.
   f. Flush main line with ˜200 mL diluent to Waste. Prime to “fill start” mark of 5 L final formulation bag.
   g. Tare 5 L bag, then add each protein from intermediate bags to formulation container (1:1:1). Start with closest intermediate bag to formulation in order from right to left (3a→2a→1a).
   h. Without flushing, tare and add required amount of diluent and seal connections on the main line.
   i. Mix adjuvant in bag for at least 30 minutes on the Wave Mixer. Weld the line to a clean line on the formulation bag.
   j. Prime line with adjuvant to fill start mark. Tare and top-up formulation bag with adjuvant.
   k. Seal lines.
   l. Seal connections and stir using Wave mixer at 20 rpm, 10 degrees for 30 min.
7) Sampling
   a. Sample up to 4×50 mL using sample bags. Sampling must be done when formulation bulk is in suspension. Ensure sampling line is flushed into a waste container (last 50 mL bag) prior to each in-series sample time point.
   b. Sampling will be done in gamma irradiated sampling bags. From these bags, they will be loaded aseptically into standard 3 mL serum vials at sampling 3× each under a GLP hood (except for container integrity) at a volume of approximately 2.5 mL/vial.
   c. At minimum, aluminum content and protein concentration will be tested for each sample point.

HPLC was performed at two laboratories, and there were differences in the results obtained from these labs because the assays and standards are neither identical nor validated (e.g. in the case of AlOOH adjuvanted proteins and Protein C protein formulations). Desorption issues surround the Protein C formulations so these results have been omitted. For the intermediate formulations, HPLC resulted in values for both intermediate formulations were within a ±30% range. For Protein D, the results for the 400 μg/mL target values vary up to ±20% (average 322 μg/mL) while the Formulations lab results vary by up to ±7% (average 427 μg/mL). For Protein A, the results for the 400 μg/mL target values vary by ±4% (average 387 μg/mL) while the Formulations lab results vary by up to ±5% (average 418 μg/mL).

Based on the HPLC results, values for final formulations were within a criteria range of ±30% for the Bivalent formulation. The lab results for the 20 μg/mL/protein target values vary by ±15%. Results for these samples were: Protein A=19 μg/mL and Protein D=23 μg/mL. The results for the 20 μg/mL/protein target values vary by up to ±21%. Results for these samples were: Protein A=18 μg/mL and Protein D=24 μg/mL. From these results, it can be concluded that the Bivalent final formulation process was successful based on accuracy as it was within a final formulation criteria of ±30%.

For GMP clinical lot implementation, a risk-based approach may be followed, taking the following aspects of a multivalent formulation process based on quality, purity, operator safety, product identity and sterility into consideration:
Omitting vent filter integrity testing of in-process filters (e.g. intermediate containers, waste). Currently, a very time-consuming step that may not be value-added.
Operator error due to complex process, lack of training
Percent (%) error and variation of protein assay methodolgy (HPLC, BCA) varies from site to site and makes it difficult to confirm in-process and final concentration of individual antigens
Pressure build-up in system may be a safety hazard or lead to back-flushing
Unexpected aggregation of intermediates or final formulated bulk
Robustness and repeatability of process
Flashing from tube welding and causing re-welding of wetted tubing lines
Wave mixer safety hazards (e.g. pinching of fingers)

### Example 11

### Broth Formulation Process

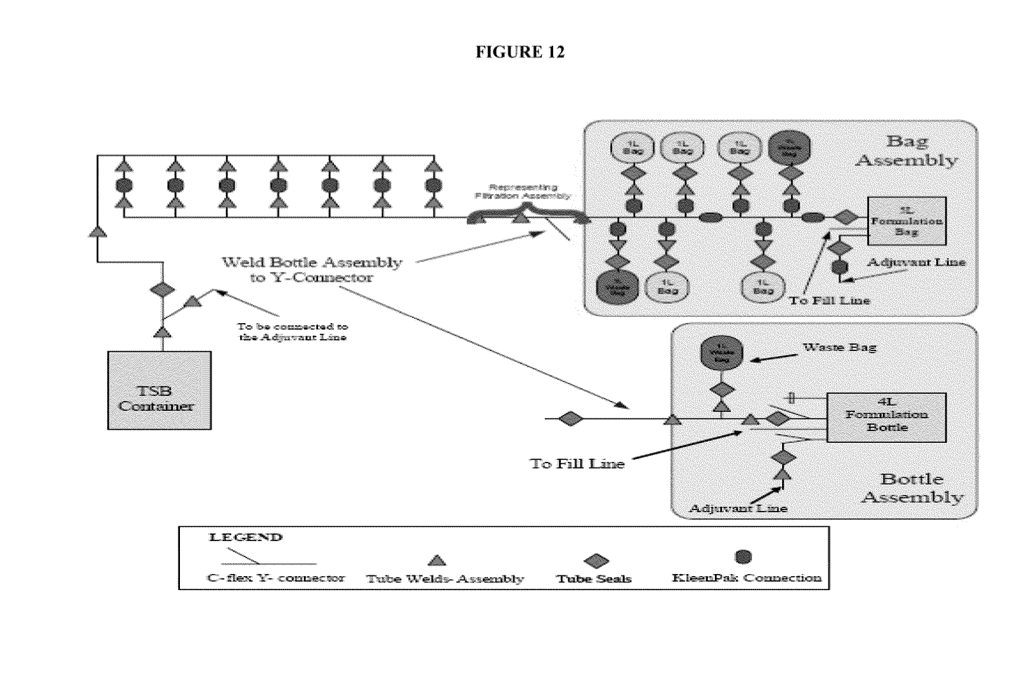

A broth formulation process was designed using the disposable bag assembly (or glass bottles as a backup) as a worst case to validate the multivalent formulation that encompasses the following: single-antigen formulation, multi-antigen formulation, intermediate formulations, sampling, dilutions, filtration formulations with treated adjuvanted, untreated adjuvanted and unadjuvanted. The worst case includes the maximum number of tube welds and seals compared to actual processes used. In addition it will include sterile connectors (e.g. Pall Kleenpak®). A diagram of an exemplary configuration is shown in FIG. 12. Tryptic Soy Broth (TSB) is passed through each line, challenging the assembly lines representing product ingredients and bags.

### Conclusions Derived from Examples 1-11

To summarize, a suitable disposables formulation process has been designed for a multivalent final bulk product with the following conclusions:
Intermediate and final bulk formulation adjuvant homogeneity is within ±0.1 mg Al/0.5 mL.
Each individual antigen is preadsorbed with AlOOH successfully as a stock intermediate supply within desirable protein concentrations (±30% or better).
Performance of a pre-adsorbed intermediate step before final blending is as or more accurate as filtering, blending then adjuvanting purified antigens in one step.
Particle size distribution of adjuvanted formulations is within expected ranges.
Wave Mixer is most efficient mixing for intermediate and final bulk product during formulation, however, mixing will be performed with a stir plate for mixing intermediates during final formulation so that mixing and dispensing of the 1 L bags can occur simultaneously.
Using a 2× Sartopore 2 filtration as primary filter assembly of choice due to reduced flushing volumes (2× Millipak 20 s and 2× EBV filters as alternatives) and confirmation of overall performance.
Flushing filtration and disposables line assembly with at least 150 mL buffer between antigen addition to intermediates is required.
No quantifiable amounts of process-induced residual proteins were detected in the single-antigen intermediate formulation using an HPLC indicating assay
No specific protein order is required for filtration using the Sartopore 2 filters at full scale based on performance studies (none required for Sartopore 2 filters)

### Example 12

Studies were performed in order to optimize the formulation and filling processes for an example Trivalent composition of proteins from Streptococcus pneumoniae (PhtD+PcpA+PlyD1) products prior to manufacturing of the toxicological lots. The goal of these studies was to determine the most efficient parameters during the formulation and the filling to ensure the final product is sterile and the concentrations are within acceptable ranges. The TBS buffer was formulated at pH 7.4, with 50 mM Tris and 150 mM NaCl. The antigens were PcpA, PhtD and PlyD1. PcpA and PhtD are in solution in the TBS buffer. PlyD1 purified bulk antigen is supplied in solution in TBS buffer with residual Tween 80 (0.05%). The phosphate-treated hydroxide (PTH) aluminum adjuvant (AlPO₄) contained 5.6 mg Al/mL and 2 mM NaPO₄, in solution in the TBS buffer. The process was performed in a closed, disposable assembly. The assembly was considered closed downstream of the final filter. The assembly is pre-sterilized as provided by the manufacturer.

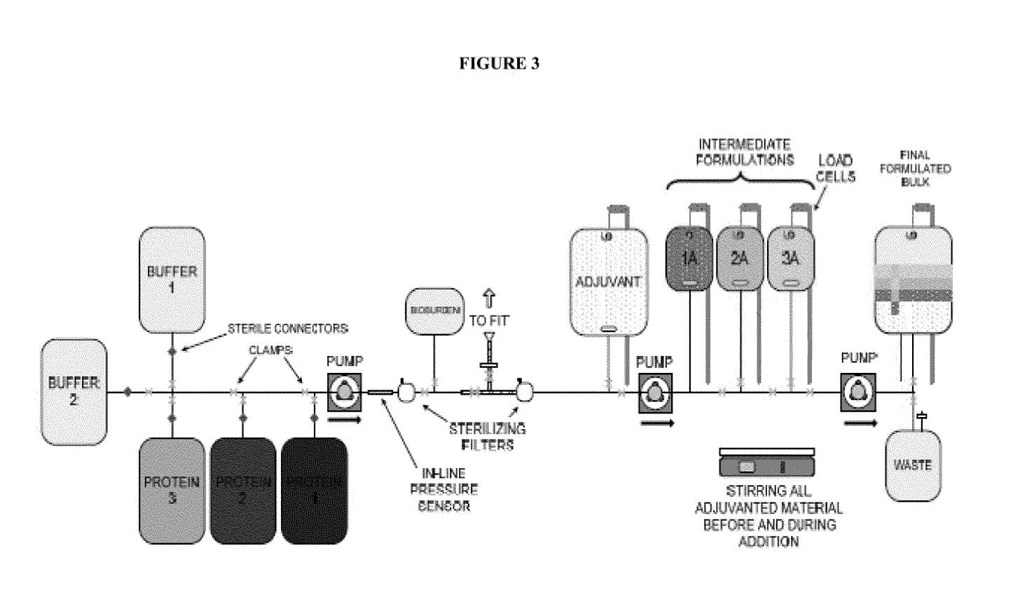

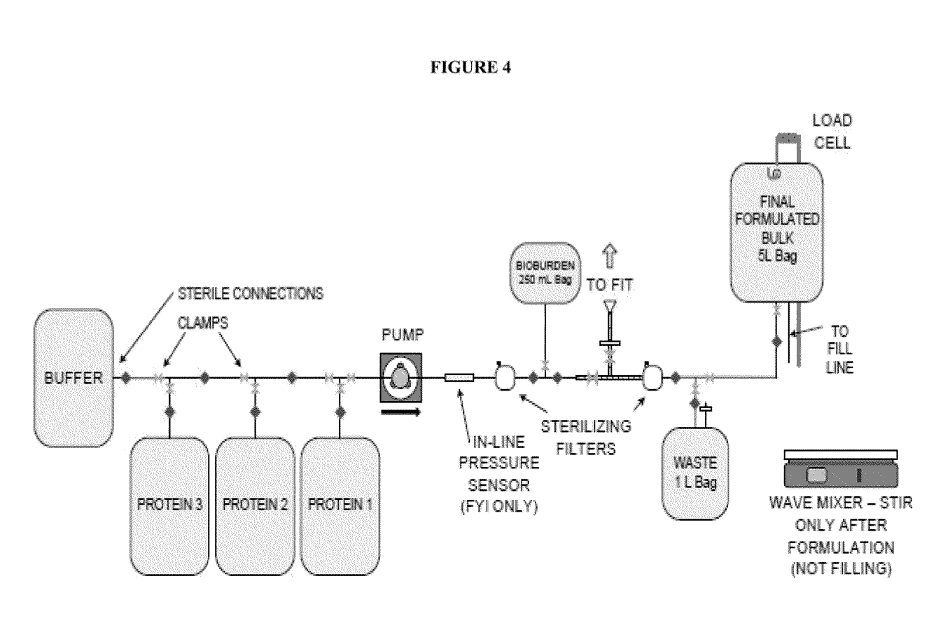

Optimization and toxicity lot testing was carried out using the systems described in FIG. 3 (AlPO₄-adjuvanted formulation) and FIG. 4 (unadjuvanted formulation). Kleenpak sterile connectors were used in-process where possible (e.g., for buffer addition, see diamond-shaped connectors in FIGS. 3 and 4). As shown in FIG. 3, the adjuvant source was relocated from near the final formulated bulk bag to before the intermediate bags to allow for unidirectional pumping. Adjuvant concentrations were also increased from 0.56 mg Al/mL in the intermediates to eliminate addition of adjuvant to the final bulk formulation to compensate for dilution (FIG. 3). The waste bag was also positioned at the end of the process line in order for correct fluid displacement and ingredient addition (FIG. 3). A Pendotech pre-sterile in-line pressure sensor (supplied by Pall) to measure the pressure pre-filtration for indication of clogging and/or line blockage (FIGS. 3 and 4). The bioburden bag from before the redundant (first filter) to between the first and second filter as this is more compliant with the regulatory guidelines as the sample is pulled just prior to the, final sterilizing filter (FIGS. 3 and 4). The load cell apparatus (FIG. 3) was utilized as “stand-alone” system that was not affixed to the formulation table to prevent unwanted vibrations or interference, provide improved stability, and a single control panel was used, instead of one control panel for each cell.

Regarding the filterability studies, any of the following five filters are suitable for the sterile filtration of a trivalent or monovalent PhtD, PcpA, or PlyD1 product: Sartorius Sartopore 2, Pall EDF, Pall EBV, Pall EKV, Millipore Millipak 20 (Table 18). Sartopore 2 was selected as the filter of choice. The Sartopore 2 filters challenged with B. diminuta in solution in the Trivalent product were able to retain 100% of the bacteria. Thus this filter can be safely used for the sterile filtration of the trivalent product. The minimum bubble point was previously tested in an MTECH report (C#010578) with the same buffer flushing solution (TBS) as the wetting agent and is 30. The maximum bubble point was recommended as 55 PSI by MTECH, however, at times this can be exceeded due to the surface tension of the buffer on the filter. A maximum parameter of ≧55 PSI is now indicated in the batch production records for the trivalent and related products.

TABLE 18

| Filters | SHF | SHC | Millipak20 | Sartopore2 | EBV | EKV | EDF |
|---|---|---|---|---|---|---|---|
| Number of | 1 | 2 | 1 | 2 | 2 | 2 | 2 |
| layers | | | | | | | |
| First layer | PES | PES | PVDF | PES | PES | PES | PES |
| material | | | | | | | |
| First layer | 0.22 μm | 0.5 μm | 0.22 μm | 0.45 μm | 0.45 μm | 0.65 μm | 0.45 μm |
| pore size | | | | | | | |
| Second layer | — | PES | — | PES | PES | PES | PVDF |
| material | | | | | | | |
| Second layer | — | 0.22 μm | — | 0.22 μm | 0.22 μm | 0.22 μm | 0.22 μm |
| pore size | | | | | | | |

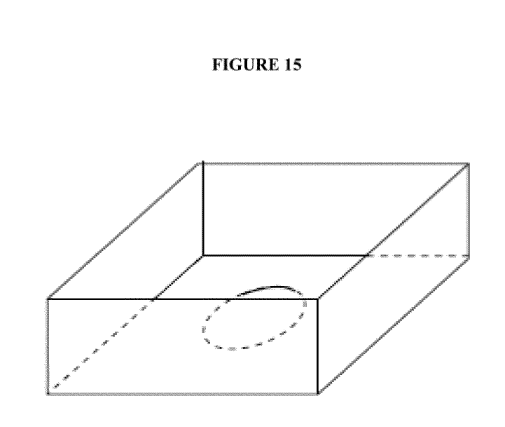

The mixing studies showed that the best parameters to maintain a trivalent adjuvanted product homogeneous are 350 rpm, with proper purging for bottom bag samples, for at least 30 minutes for an adjuvanted product formulated in a 3 L-3 D bag. A clear plastic bag holder (FIG. 15; other materials may also be used, such as stainless steel) with a hole at the bottom was used to prevent the bag from moving on the surface of the magnetic stirrer and so that visibility of settling and volume levels is possible. The aggregation studies indicated that a magnetic stirrer induces more aggregation than a WaveMixer on a trivalent unadjuvanted product. The particulates are approximately 10 times bigger. However, the aggregated particulates are not visible and do not induce any variation in the protein concentration. Mixing conditions of unadjuvanted product on the WaveMixer are 20 rpm, 10° for a minimum of 10 minutes.

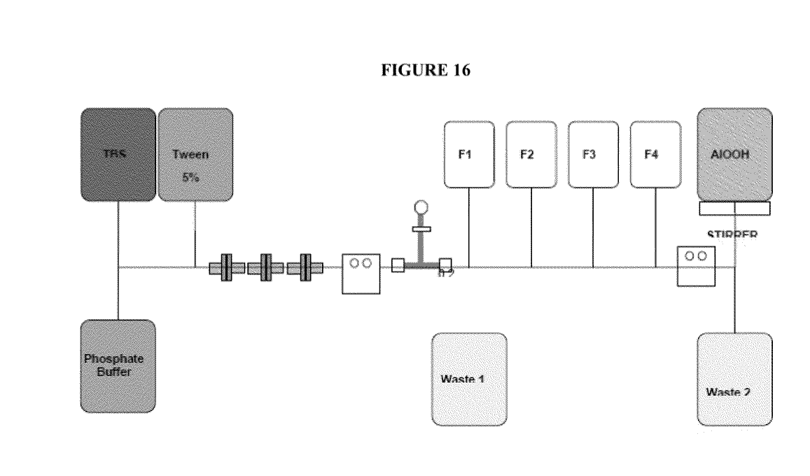

Leachables studies were performed on the TC-Tech bags with several ingredients including stock Aluminum Hydroxide Adjuvant, Phosphate Treated Hydroxide Adjuvant, Tween-containing product and representative final product using the system shown in FIG. 16. Bags containing 0.05% Tween 80 showed an unidentified peak that may have been Tween related or a leachable from the bag due to the presence of Tween. Bags, representing final product with residual Tween 80, containing 0.025% Tween 80 in TBS and Phosphate Treated Hydroxide, showed no peaks up to 1 month. This was consistent with findings from an MTECH study clone on the bags using a higher concentration of Tween (0.5%, 5000 ppm, C012431). After 6 months, for the stock phosphate treated hydroxide or final product without Tween 80 residual, one unidentified peak was detected by HPLC/UV. All other results showed no noncarcinogenic, non-toxic leachables above reportable values.

The optimization runs showed that the mixing and formulation parameters were suitable to maintain the proteins and aluminum concentrations stable throughout the filling process. Some investigations were performed to determine low protein and aluminum concentrations detected in some lots were related to the assay methodology or change of columns. The product appearance, based on visual inspection performed during the optimization and toxicity lot runs is described as clear, colorless solution for unadjuvanted Trivalent product, and a white, cloudy suspension for adjuvanted Trivalent product. Five optimization runs with Trivalent products were conducted. The optimization runs were performed with a Trivalent unadjuvanted product and the Trivalent adjuvanted products. Two optimization runs at full scale with adjuvanted, high dose (100 μg/mL/protein, PhtD+PcpA+PlyD1) mixed in formulated bulk in a 3 D 3 L bag for final blending (min 30 minutes) and filled (minimum 30 minutes) at 350 rpm on a stir plate. Two optimization runs were also performed using unadjuvanted, high dose (100 μg/mL/protein, PhtD+PcpA+PlyD1) mixed in formulated bulk in 5 L a bag for final blending (min 10 minutes) using 20 rpm, 10° on a WaveMixer. Optimization runs for adjuvanted, low dose (20 μg/mL/protein, PhtD+PcpA+PlyD1) mixed as a formulated bulk in 3 D 3 L bag for final blendine. (minimum 30 minutes) and filled (minimum 30 minutes) using 350 rpm on stir plate. The formulated bulk and filled vials were analyzed for protein content and, where applicable, aluminum and phosphorous content. Samples were analyzed for protein content by HPLC, with an expected target range for high dose is 70-130 μg/mL and for low dose is 14-26 μg/mL. Samples were analyzed for aluminum and phosphorous content by with an expected target range for aluminum content was 0.56±0.2 mg Al/mL. Samples were also checked for visual inspection to define an appropriate description for product appearance for both adjuvanted and unadjuvanted trivalent products. The high dose formulations are representative of high, medium and low dose. The samples were analyzed for protein content by HPLC, where applicable though it was found that these samples were difficult to pull as there was insufficient representative material in the intermediates that remained after final blending. Samples were also taken during filling at the beginning, middle and end to be analyzed for protein content to ensure the homogeneity of the formulated bulk. Samples were also taken from the beginning and the end of the filling process to be analyzed for aluminum content and phosphorous content to ensure homogeneity.

The protein concentrations for the unadjuvanted trivalent high dose formulations were acceptable, although the concentration for PhtD was low in one instance PlyD1 was deemed as not a reportable value. For these samples, the protein concentrations were consistent throughout the beginning, middle and end filling samples tested indicating no adverse trending during filling of unadjuvanted product.

The protein concentrations for the adjuvanted trivalent high dose formulations were acceptable. The aluminum content for these samples were acceptable for beginning and end samples; phosphorous was reported at about 2.2-2.3 mM (trending only; one sample was reported as low for unknown reasons). For these samples, the protein concentrations were consistent throughout the beginning, middle and end filling samples tested indicating no adverse trending during filling of unadjuvanted product. The protein and adjuvant concentrations were consistent throughout the filling process indicating product homogeneity is maintained during mixing and filling.

Table 19 shows the results obtained for the different optimization and toxicity runs for protein content, aluminum content and phosphorous content.

TABLE 19

| | | | intermediate | final formulation | | Aluminum Content | | | Phosphorous Content | |
|---|---|---|---|---|---|---|---|---|---|---|
| | | | intermediate | concentration | concentration | mg Al/mL | | | mM | |
| | protein | lot # | concentration | F&S | ASAD | | QC | Exova | | Exova |
| CA-09- | PcpA | 09-T-DP010 | | 97 | 100 | B | | | B | |
| 136-A | PhtD | 08-T-DP014 | | 74 | 73 | M | | | M | |
| (Unadj) | PlyD1 | 08-T-DP013 | | 82 | | E | | | E | |
| CA-09- | PcpA | 09-T-DP009 | | 109 | 111 | B | | | B | |
| 136-D | PhtD | CDP0020 | | 112 | 108 | M | | | M | |
| (Unadj) | PlyD1 | 08-T-DP017 | | 112 | 116 | E | | | E | |
| CA-09- | PcpA | 09-T-DP010 | 419 | 101 | 107 & 113* | B | | 0.52 | B | 2.16 |
| 136-B | PhtD | 09-T-DP002 | 395 | 93 | 75.9 & 92* | M | | | M | |
| (Adj) | PlyD1 | 08-T-DP012 | 444 | 94 | NR & 108* | E | | 0.52 | E | 2.16 |
| CA-09- | PcpA | 09-T-DP010 | 399 | 100 | 106 | B | 0.57 | 0.43** | B | 2.30 |
| 136-C | PhtD | CDP0020 | 413 | 94 | 86 | M | 0.56 | | M | |
| (Adj) | PlyD1 | 08-T-DP017 | 557 | 115 | | E | 0.57 | 0.45** | E | 2.31 |
| 09-T- | PcpA | 09-T-DP013 | | 105 | 103 | B | 0.62 | 0.56 | B | 2.45 |
| FS016 | PhtD | 09-T-DP027 | | 91 | 92 | M | 0.62 | | M | |
| (Adj Tox) | PlyD1 | CDP0030 | | 127 | 120 | E | 0.62 | 0.56 | E | 2.45 |
| 09-T- | PcpA | 09-T-DP013 | | 109 | 109 | B | | | B | |
| FS014 | PhtD | 09-T-DP027 | | 95 | 96 | M | | | M | |
| (Unadj | PlyD1 | CDP0030 | | 115 | 114 | E | | | E | |
| Tox) | | | | | | | | | | |

*Sent as a blind sample (labelled as 136E)
**Initial results (Retest performed; Investigation report pending)
NR = Result not reported

### Example 13

### Trivalent Adjuvanted Formulation Process in Closed Disposable Assembly

The procedure described in this Example shows that, at least for the combination of PhtD, PcpA, and PlyD1, the antigens may be combined directly with adjuvant without preparing an intermediate formulation. The reagents used in these procedures were: PhtD purified concentrated bulk; PcpA purified concentrated bulk; PlyD1 purified concentrated bulk; Tris-HCl buffered Saline (TBS) pH 7.4, 10 mM Tris, 150 mM NaCl; Adjuvant: 5.6 mg Al/mL (consisting of AlOOH adjuvant at 9-11 mg Al/mL starting concentration, sodium phosphate buffer (400 mM), and Tris-HCl Buffered Saline (TBS) pH 7.4, 10 mM Tris, 150 mM NaCl).

The TC-Tech formulation assemblies where the formulations are performed include two (2) Sartopore 2 filters with filter integrity system, 1 L bags with C-flex 072 tubing, 3 L-3 D bags with C-flex 072 tubing, and C flex 072 Y connectors.

Three studies were conducted at laboratory scale. In study CA-09-212, a trivalent composition was formulated by adding the individual protein into 2 mM adjuvant and finally diluted with 10 mM TBS buffer pH 7.4. A 15 min mixing was conducted after the addition of each ingredient. In study CA-09-226 the proteins were added sequentially to the formulation bottle (125 mL Nalgene bottle), mixed and finally the adjuvant was added to the mixture of antigens. Final dilution to required concentration and mixing was done with TBS buffer. In study CA-10-007 the proteins were added sequentially to the formulation bottle (125 mL Nalgene bottle), then adjuvant and finally TBS. As compared to CA-09-226, the CA-10-007 study included only one mixing step, which was conducted after all ingredients were added to the formulation container. Additionally, different mixing times were evaluated (i.e., from 1 min to up to 18 h). Critical parameters that were measured included % Adsorption; Protein content and Aluminum content. The results from these three studies were compared to formulations produced using intermediate bulk (i.e., studies CA-09-084, CA-10-009, CA-09-146).

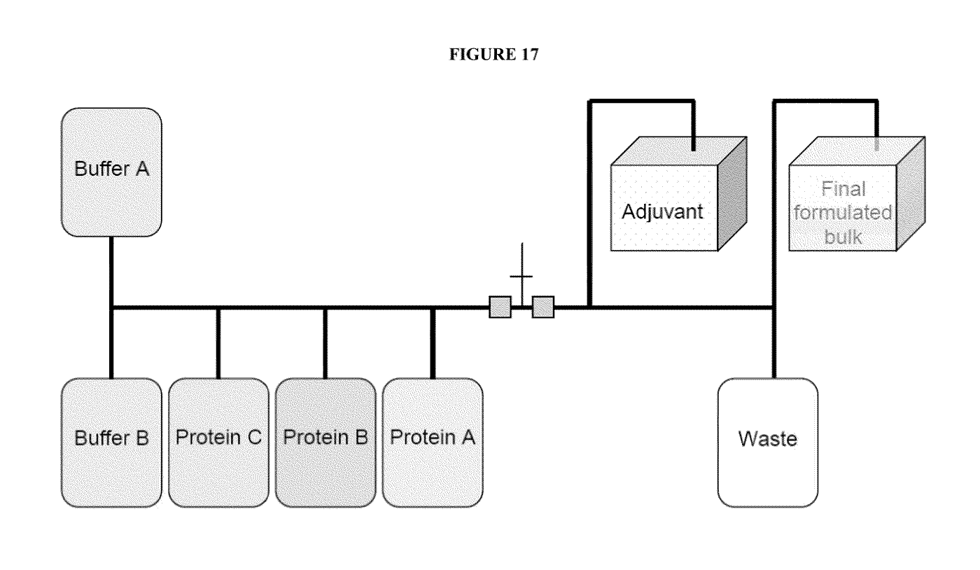

Three different trivalent adjuvanted high dose 3 L lots formulated using a process without intermediates were prepared. The equipment used to produce these formulations is illustrated in FIG. 17. The adjuvant bag is filled beforehand with the correct amount of AlOOH. The process may be (and was) carried out as follows:
1. Prime each protein and the phosphate buffer down to the T-junction. Ensure all air in the line is displaced.
2. Flush the main line with 200 g of TBS to the waste bag. Ensure the filters are wet and the filter capsules are full of TBS.
3. Prime the adjuvant bag with TBS to the fill-start mark.
4. Prime each intermediate bag with TBS to the fill-start mark.
5. Prime the final formulation with TBS to the fill-start mark.
6. Tare the adjuvant bag. Add the required amount of phosphate to the adjuvant bag.
7. Tare the adjuvant bag. Add the required amount of TBS to the adjuvant bag.
8. Mix the adjuvant 10× bag on a magnetic stirrer for at least 30 minutes at 350 rpm.
9. Flush 200 g of adjuvant to the waste bag.
10. Tare the final formulation bag and add the required amount of adjuvant to the bag.
11. Flush the main line with 200 g of diluent to the waste bag.
12. Tare the final formulation bag and add the required amount of the protein closest to the filter.
13. Tare the final formulation bag and add the required amount of the second protein.
14. Tare the final formulation bag and add the required amount of the third protein.
15. Tare the final formulation bag and add the required amount of diluent to the bag.
16. Mix the final formulation bag on a magnetic stirrer for 30 minutes at 350 rpm.

Samples were taken from the final formulation bag after mixing and were analyzed for protein content, aluminium content, phosphorus content and percentage of adsorption of each protein on the adjuvant. A 30-minute mixing time was previously shown to be efficient. In order to determine whether a shorter mixing time would have the same efficiency and therefore save some time in the formulation process, samples were also taken after 15-minute mixing. These samples were analyzed and compared with the samples taken after 30-minute mixing.

Particle size was measured by using a Mastersizer 2000 linked to a Hydro 2000S sample dispersion unit both from Malvern Instruments. The particle size was measured by laser diffraction granulometry. The results were processed by volume and the data utilized for characterization was d(0.5): diameter below which 50% of the particles are distributed by volume (d(0.1) and d(0.9).

The results from the small scale studies were compared with previous trivalent formulations produced with intermediate bulks. The first simplified process at small scale involved the sequential addition of protein antigens to the final container containing the adjuvant. After the addition of each protein, a 15 min mixing was conducted with a final 30 min mixing after the addition of dilution buffer. In comparison to formulations prepared using intermediate bulks, adsorption and protein concentration results from these three batches produced by this process showed no significant differences. Aluminum and phosphorous content were within the expected limits indicating comparability among the two processes using these antigens. These results suggest that intermediate adsorbed antigens may not be required to maintain optimal adsorption to the adjuvant in the final formulation.

In a second round of experiments, formulations were prepared without intermediates by adding first adjuvant and then the individual antigens. The goal was to investigate whether the order of addition of ingredients significantly affected quality parameters of the formulation. Additionally, the mixing step after the addition of each ingredient was eliminated to reduce the formulation time.

Finally, in study CA-10-007 the effect of mixing time duration was investigated to evaluate the adsorption kinetics of the antigens to the adjuvant (AlOOH). In these experiments mixing was conducted at the end of the formulation process for different durations (from 5 min to up to 18 h). Almost 100% adsorption to AlOOH was detected in less than 5 min. These results suggest fast binding kinetics for all three antigens. The results also indicate that continuous mixing at low speed does not significantly affect the % adsorption or protein content in the trivalent formulation (Table 20).

TABLE 20
Aluminum and phosphorous content in Trivalent
formulations produced without intermediates*

| | P (mmol/L) | Al (mg/ml) |
|---|---|---|
| PPrV Trivalent Lot # | (1-3 mM) ⁽¹⁾ | (0.36-0.76 mg/ml) ⁽²⁾ |
| CA-09-212-A | 2.0 | 0.50 |
| CA-09-212-B | 2.0 | 0.49 |
| CA-09-212-C | 2.0 | 0.47 |
| | Average: 2.0 | Average: 0.49 |
| | RSD: 0% | RSD: 3.14% |

*Proposed release limits for P ⁽¹⁾ and Al ⁽²⁾ are indicated.

P Consistency in lot manufacturing can be monitored by measuring particle size. The particle size of the 3 L formulations produced by the two different processes was evaluated by laser diffraction granulometry for small scale formulations. No significant differences were observed in particle size of the adjuvanted formulations produced without intermediates suggesting that the lack of intermediates has no influence on the particle size of the adjuvant using this combination of antigens.

All references cited and/or listed herein are hereby incorporated into this disclosure in their entirety. While certain embodiments have been described in terms of the preferred embodiments, it is understood that variations and modifications will occur to those skilled in the art. Therefore, it is intended that the appended claims cover all such equivalent variations that come within the scope of the following claims.

### REFERENCES

Cardona, et al. Filtration Designs Remove Processine Bottlenecks for High-Yield Biotech Drugs, Supplement to Biopharm International, June 2006.
Cardona, et al. Incorporating Single-Use Systems in Biopharmaceutical Manufacturing, Bioprocess International, Disposables Supplement, June 2006.
Roberson, et al. Engineering Fluid Mechanics, Sixth Edition, Wiley, 1997
Motzkau, et al. The Importance of Vendor Validation Services: Experience and Economics, BioProcess International, September 2005.
Sharma, et al. Filter Clogging Issues in Sterile Filtration, Biopharm International, April 2008
Baumfalk, et al. Integrity Testing in the Pharmaceutical Process Environment, BioPharm International, Volume 19, Number 6, June 2006
Priebe, et al. Choosing and Scaling Up Right Filter Combo, Bioprocessing Tutorial, Genetic Engineering News, Mar. 1, 2006
Luckiewicz, E. Elements of Applied Process Engineering Course Notes, Center for Professional Advancement, New Brunswick, N.J., March 2004
Doran, P. Bioprocess Engineering Principles, Academic Press, 1995
Cardona, M. Considerations for Buffer Filtration, Contamination Control, June/July 2005
EMEA, Manufacture of the Finished Dosage Form (Directive 81/852/EEC as amended), December 1995
US Food and Drug Administration. Sterile Drug Products Produced By Aseptic Processing, Current Good Manufacturing Practices (Guidance for Industry), September 2004
ICH Harmonised. Tripartite Guideline, Quality Risk Management Q9, Current Step 4 version, November 2005
Phillips, C. It's Not Whether but Rather What and How to Implement, Bioprocess International, May 2008
Singh, V. BioProcess Tutorial, Non-invasive mixing in bags, February 2000
Sharma, et al. Filter clogging issues in sterile filtration, Biopharm Internation, April 2008

1. A sterile, closed, disposable system for formulating a biopharmaceutical composition comprising multiple active agents, the system comprising:
(a) one or more buffer reservoirs;
(b) multiple reservoirs of active agents, each;reservoir containing a different active agent or combination of active agents;
(c) one or more pumps;
(d) one or more sterilizing filters;
(e) a station for mixing the formulations with one another, the station comprising:
1) at least one intermediate formulation reservoir corresponding to each reservoirs in (b);
2) optionally at least one auxiliary reservoir containing one or more additional components;
3) at least one pump for combining the contents of each load cell and the auxiliary reservoir in a final bulk formulation reservoir;
wherein parts (a) through (e) are operably linked to one another in series.

2. The system of claim 1 wherein each of the reservoirs of (b) is a single-use, pre-sterilized bag.

3. The system of claim 1 comprising at least two sterilizing filters.

4. The system of claim 3 further comprising a bioburden container positioned between the at least two sterilizing filters.

5. The system of claim 1 further comprising a waste container positioned between the at least one sterilizing filter and the final bulk formulation reservoir.

6. The system of claim 1 further comprising part (f), part (f) being a waste container positioned at the end of the process line after the station of (e).

7. The system of claim 1 wherein the station of (e) is not fixably attached to a support surface.

8. The system of claim 1 wherein each active agent of (b) is an antigen and the one or more additional components is an adjuvant.

9. A method for preparing a multi-component biopharmaceutical composition comprising combining multiple active agents from individual active agents contained in individual reservoirs after passing the contents of each reservoir through at least one sterilizing filter, combining the components of each reservoir into an intermediate formulation within a container containing one or more additional components, and combining the contents of each container into a final formulation comprising all active agents and additional components.

10. The method of claim 3 wherein the one or more additional components is an adjuvant.

11. A sterile, closed, disposable system for formulating a biopharmaceutical composition comprising multiple active agents, the system comprising:
(a) one or more buffer reservoirs;
(b) multiple reservoirs of active agents, each reservoir containing a different active agent or combination of active agents;
(c) one or more pumps;
(d) one or more sterilizing filters;
(e) a station for mixing the multiple active agents with one another, the station comprising:
1. a final bulk formulation reservoir;
2. optionally, at least one auxiliary reservoir containing one or more additional components;
3. at least one pump for combining the contents of each active agent reservoir and the auxiliary reservoir in the final bulk formulation reservoir;
wherein parts (a) through (e) are operably linked to one another in series.

12. The system of claim 11 wherein the system comprises (e) a station for mixing formulations, the station comprising:
1) at least one intermediate formulation reservoir corresponding to each of the reservoirs in (b);
2) optionally, at least one auxiliary reservoir containing one or more additional components; and
3) at least one pump for combining in each intermediate formulation reservoir the contents of the corresponding active agent reservoir with the contents of the auxiliary reservoir and for later, combining the contents of each intermediate formulation reservoir in a final formulation reservoir;
wherein parts (a) through (e) are operably linked to one another in series.

13. The system of claim 11 wherein each of the reservoirs of (b) is a single-use, pre-sterilized bag.

14. The system of claim 11, wherein parts (a) through (e) are operably link to one another by pre-sterilized tubing.

15. The system of claim 11 comprising at least two sterilizing filters.

16. The system of claim 15 further comprising a bioburden container positioned between the at least two sterilizing filters.

17. The system of claim 11 further comprising a waste container positioned between the at least one sterilizing filter and the final bulk formulation reservoir.

18. The system of claim 11 further comprising part (f), part (f) being a waste container positioned at the end of the process line after the station of (e).

19. The system of claim 11 wherein the station of (e) is not fixably attached to a support surface.

20. The system of claim 11 wherein each active agent of (b) is an antigen and the one or more additional components is an adjuvant.

21. A sterile, closed system comprising one or more first reservoirs containing the same or different buffers, one or more second reservoirs containing the same or different antigens, at least one third reservoir containing at least one adjuvant, the reservoirs being linked in series wherein the at least one third reservoir terminates the series and samples from each reservoir are combined to form an immunogenic composition.

22. The system of claim 21 wherein the reservoirs of a single-use, disposable material.

23. The system of claim 11 wherein the antigens are derived from a source selected from the group consisting of one or more viruses, bacterial species, fungal species, parasitic species, and tumor cell.

24. The method of claim 9 wherein the antigens are derived from a source selected from the group consisting of one or more viruses, bacterial species, fungal species, parasitic species, and tumor cell.

25. An immunogenic composition prepared using the system of claim 1.

26. The immunogenic composition of claim 25 suitable for use as a vaccine.

27. An immunogenic composition prepared by the method of claim 9.

28. The immunogenic composition of claim 27 suitable for use as a vaccine.
